SEQUENCE LISTING (28 sequences)

SEQ ID NO: 1 - DNA, 558 bp, Homo sapiens
atgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttag

SEQ ID NO: 2 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - DNA, 60 bp, Homo sapiens
gctcggttggatgtcgcgtcggagtttcgaaagaagtggaataagtgggctctgagtcgt

SEQ ID NO: 4 - protein, 20 aa, Homo sapiens
ARLDVASEFRKKWNKWALSR

SEQ ID NO: 5 - DNA, 27 bp, Homo sapiens
aagtggaataagtgggctctgagtcgt

SEQ ID NO: 6 - protein, 9 aa, Homo sapiens
KWNKWALSR

SEQ ID NO: 7 - DNA, 1457 bp, Homo sapiens
ggcacgagctggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctcccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaacc

SEQ ID NO: 8 - protein, 20 aa, Rattus rattus
ARLDTSSQFRKKWNKWALSR

SEQ ID NO: 9 - protein, 20 aa, Mus musculus
AGPDTPSQFRKKWNKWALSR

SEQ ID NO: 10 - DNA, 411 bp, Artificial Sequence
  Synthetic DNA
atgaaatgcagctgggtcatcttcttcctgatggcagtggttataggaatcaaatcagaggttcagctgcagcagtctggggcagagcttgtgaggtcaggggcctcagtcaagttgtcctgcacagcttctggcttcaatattaaagactactatatacactgggtgaaacaggggcctgaacagggcctcgagtgggttggatacattgatcctgagaatggtgagactgcatatgccccgaagttccagggcaaggccactttgactgcagacacatcctccaacacagcctacctgcagctcatcagcctgacatctgaggacactgccgtctattactgtaatgctccttatttctccctagggcggaactactggggccaaggcaccactctcacagtctcctca

SEQ ID NO: 11 - protein, 137 aa, Artificial Sequence
  Synthetic amino acid
MKCSWVIFFLMAVVIGIKSEVQLQQSGAELVRSGASVKLSCTASGFNIKDYYIHWVKQGPEQGLEWVGYIDPENGETAYAPKFQGKATLTADTSSNTAYLQLISLTSEDTAVYYCNAPYFSLGRNYWGQGTTLTVSS

SEQ ID NO: 12 - DNA, 393 bp, Artificial Sequence
  Synthetic DNA
atgaagttgcctgttaggctgttggtgctgatgttctggattcctgcttccagcagtaatgttttgatgacccaaactccactctccctgcctgtcagtcttggagatcaagcctccatctcttgcagatctagtcagagcattgtacatggtaatggagacacctatttagaatggtacctgcagaaaccaggccagtctccaaagctcctgatctacaaagtttccaaccggttttctggggtcccagacaggttcagtggcagtgggtcagggacagatttcacactcaagatcagaagagtggaggctgaggatctgggagtttattactgctttcaaggttcacatgttccgctcacgttcggtgctgggaccaagctggagctgaaa

SEQ ID NO: 13 - protein, 131 aa, Artificial Sequence
  Synthetic amino acid
MKLPVRLLVLMFWIPASSSNVLMTQTPLSLPVSLGDQASISCRSSQSIVHGNGDTYLEWYLQKPGQSPKLLIYKVSNRFSGVPDRFSGSGSGTDFTLKIRRVEAEDLGVYYCFQGSHVPLTFGAGTKLELK

SEQ ID NO: 14 - protein, 5 aa, Artificial Sequence
  Synthetic peptide
DYYIH

SEQ ID NO: 15 - protein, 17 aa, Artificial Sequence
  Synthetic peptide
YIDPENGETAYAPKFQG

SEQ ID NO: 16 - protein, 9 aa, Artificial Sequence
  Synthetic peptide
PYFSLGRNY

SEQ ID NO: 17 - protein, 16 aa, Artificial Sequence
  Synthetic peptide
RSSQSIVHGNGDTYLE

SEQ ID NO: 18 - protein, 7 aa, Artificial Sequence
  Synthetic peptide
KVSNRFS

SEQ ID NO: 19 - protein, 9 aa, Artificial Sequence
  Synthetic peptide
FQGSHVPLT

SEQ ID NO: 20 - DNA, 27 bp, Artificial Sequence
  Synthetic primer
tttttttttttttttttttttttttnn

SEQ ID NO: 21 - DNA, 30 bp, Artificial Sequence
  Synthetic oligonucleotide
aagcagtggtatcaacgcagagtacgcggg

SEQ ID NO: 22 - DNA, 35 bp, Artificial Sequence
  Synthetic oligonucleotide
cccaagcttccagggnccannggatanacngntgg

SEQ ID NO: 23 - DNA, 30 bp, Artificial Sequence
  Synthetic oligonucleotide
cccaagcttactggatggtgggaagatgga

SEQ ID NO: 24 - DNA, 20 bp, Artificial Sequence
  Synthetic oligonucleotide
gttgtaaaacgacggccagt

SEQ ID NO: 25 - protein, 13 aa, Artificial Sequence
  Synthetic peptide
EFRKKWNKWALSR

SEQ ID NO: 26 - protein, 11 aa, Artificial Sequence
  Synthetic peptide
RKKWNKWALSR

SEQ ID NO: 27 - protein, 9 aa, Artificial Sequence
  Synthetic peptide
KWNKWALSR

SEQ ID NO: 28 - protein, 8 aa, Artificial Sequence
  Synthetic peptide
WNKWALSR

(19) United States
(12) Patent Application Publication
(10) Pub. No.: US 2011/0301332 A1
(43) Pub. Date: Dec. 8, 2011

(54) ANTI-PAMP THERAPEUTIC ANTIBODIES

(75) Inventors: David Wilson, Fremont, CA (US); Tetsuya Taura, Palo Alto, CA (US)

(73) Assignee: Cephalon Australia (VIC) Pty Ltd., Parkville, Victoria (AU)

(21) Appl. No.: 13/001,744

(22) PCT Filed: Jul. 1, 2009
(86) PCT No.: PCT/AU2009/000850

§ 371 Date: Mar. 24, 2011

Related U.S. Application Data
(60) Provisional application No. 61/077,461, filed on Jul. 1, 2008.

(51) Int. Cl. C07K 16/18; C12N 5/18

(52) U.S. Cl. 530/387.3; 530/387.9; 435/331

(57) ABSTRACT

The invention relates to antibodies and antigen-binding fragments thereof which bind to proadrenomedullin N-terminal peptide (“PAMP”). Furthermore, the invention comprises monoclonal antibody EGX-P-E9 as well as engineered variants thereof, including chimeric, humanized or de-immunized versions thereof. The antibodies of the invention are useful for inhibiting the physiological activities of PAMP as well as in diagnosis of PAMP-responsive conditions.

### FIELD OF THE INVENTION

The present invention relates to antibodies that bind to proadrenomedullin N-terminal 20 peptide (“PAMP”). More specifically, the invention relates to anti-PAMP antibodies that can be used in research, as diagnostic tools, and as therapeutic agents.

### BACKGROUND OF THE INVENTION

Proadrenomedullin N-terminal 20 peptide (“PAMP”)

### Sequence, Forms, and Expression

The proadrenomedullin N-terminal 20 peptide is a molecule originating from post-translational enzymatic processing of pre-proadrenomedullin (see, as examples, Martínez A et al; and Ishimitsu et al.); pre-proadrenomedullin (SEQ. ID NO: 2) is a 185-amino acid precursor for adrenomedullin (“AM”) and PAMP. AM is a peptide hormone having several described physiological activities, such as potent angiogenic, hypotensive, and vasodilatory effects. Biosynthesis and secretion of AM and PAMP are regulated differentially in various tissues (e.g. Matsui E. et al.). For example, the proadrenomedullin-derived peptides AM and PAMP are expressed in the pituitary in cell-specific and not overlapping patterns, which could be explained by differences in postranslational processing (Montuenga L. M. et al.); furthermore, in the prostate, AM has a similar cellular distribution pattern to that of its mRNA, but AM and PAMP are stored and secreted from different cell types (Jiménez N.). This, in combination with other significant evidence, suggests that AM and PAMP are not always co-expressed or co-secreted, though both are products of post-translational enzymatic processing of pre-proadrenomedullin, and that they play different physiological and pathophysiological roles.

TABLE 1
PAMP Sequence.

Human PAMP-20 (SEQ ID NO: 4):
N-Ala Arg Leu Asp Val Ala Ser Glu Phe Arg Lys Lys
Trp Asn Lys Trp Ala Leu Ser Arg-C
Human PAMP-12 (SEQ ID NO: 6):
N-Lys Trp Asn Lys Trp Ala Leu Ser Arg-C
Sequence comparison between human, rat, and
mouse PAMP:
(SEQ ID NO: 4)

| | |
|---|---|
| Human | N-Ala Arg Leu Asp Val Ala Ser Glu Phe Arg |
| | Lys Lys Trp Asn Lys Trp Ala Leu Ser Arg-C |

(SEQ ID NO: 8)

| | |
|---|---|
| Rat | N-Ala-Arg Leu Asp Thr Ser Ser Gln Phe Arg |
| | Lys Lys Trp Asn Lys Trp Ala Leu Ser Arg-C |

(SEQ ID NO: 9)

| | |
|---|---|
| Mouse | N-Ala Gly Pro Asp Thr Pro Ser Gln Phe Arg |
| | Lys Lys Trp Asn Lys Trp Ala Leu Ser Arg-C |

Note:
all of these sequences are amidated at the C-terminus.

PAMP is a C-terminally amidated peptide molecule consisting of the 20 N-terminal amino acids of pre-proadrenomedullin. The sequence of human PAMP (SEQ ID NO:4) is shown in Table 1. PAMP sequence homology across species is highly conserved in the C-terminal 12 amino acids, whereas the N-terminal 8 amino acids show a lesser degree of conservation (see Table 1). PAMP is also sometimes referred to as PAMP-20 in order to distinguish it from truncated analogues of the molecule that have been synthesized and studied. One such truncated version of PAMP, PAMP-12, consists of residues 12-20 of PAMP and is also amidated at the C-terminus. The sequence of human PAMP-12 (SEQ. ID NO:6) is shown in Table 1. The sequence/structure of PAMP-12 is sufficient to activate the required signaling cascades in order to affect certain physiological functions, whereas for other functions (e.g. inhibition of aldosterone and catecholamine secretion), the N-terminal amino acids are required. Intravenous injections of PAMP-12 results in a significant, dose-dependent hypotensive effect, which is comparable to the effect of PAMP-20 (Kuwasako K et al.). Amidation of the C-terminal residue of PAMP and PAMP-12 is critical for receptor-binding and downstream signaling (Iwasaki H. et al.).

PAMP is expressed at high levels in adrenal medulla and pheochromocytoma tissue (Washimine H. et al.); it expressed abundantly in the duodenum and ileum, whereby it is found in the mucosa and submucosa of the ileum at significantly higher concentrations than those in whole ileum, and it is ubiquitously expressed in other gastrointestinal tissues (Kiyomizu A. et al.). PAMP is also expressed in myocardial tissue, in the prostrate, pituitary gland and brain, and in skin (Jiménez N. et al. Ueta Y et al.), considerable concentrations of PAMP are also found in human plasma and urine (e.g. Matsui E et al; Kinoshita H. et al.).

In cardiac myocytes, PAMP expression is induced by the growth-promoting stimulation of Angiotensin II (Tsuruda T et al. 2001.). PAMP concentrations are increased in heart tissue of spontaneously hypertensive subjects; the atrial concentration of PAMP, in particular, is significantly higher than that of the normotensive subjects (Inatsu H. et al; Etoh T et al.).

In septic shock, tissue concentration of PAMP in the lung increases significantly, but decreases it in the adrenal gland and cardiac atrium (Matsui E. et al.).

In neuronal cells, NGF stimulation initially increases the rate of pre-proadrenomedullin mRNA expression for the first hour, and thereafter progressively suppresses the expression levels to one fifth of that in non-NGF-stimulated neurons over a 72 hour time-course; however, the mRNA degradation rate is also reduced by NGF stimulation, up to, and presumably beyond the 72 hour time point (Kobayashi H. et al. 2004).

Furthermore, PAMP is released along with catecholamines by regulated exocytosis upon stimulation of adrenal chromaffin cells (Kobayashi H et al. 2001).

### The PAMP Receptor and Signaling

Specific receptors sites for rat PAMP are expressed widely in various tissues at low abundance, and at higher abundance in aorta and adrenal glands, followed by lung, kidney, brain, spleen, and heart. Autoradiography showed the presence of abundant PAMP binding sites in both the outer cortex and medulla of human adrenals (Andreis P G et al.). An equilibrium binding study revealed the presence of high-affinity binding sites for PAMP (Iwasaki H et al.). It has been reported that the G protein-coupled receptor MrgX2, which belongs to the large family of the Mas-related genes or sensory neuron-specific G protein-coupled receptors, is activated by PAMP binding, and may therefore be a PAMP receptor (Nothacker H. P. et al.), although this has not been conclusively demonstrated, and there may be other PAMP receptors. Several downstream PAMP signaling events are abolished by G protein and protein kinase inhibitors (Shimosawa T et al. 1997; Qi Y F et al.).

Downstream PAMP signaling impairs Calcium influxin in various cell types (e.g. Belloni A S et al.), and treatment of vascular smooth muscle cells with PAMP also increases intracellular cAMP concentrations and inhibits transcription of pre-proAM mRNA and AM secretion (Qi Y F et al.). Upon intracerebroventricular administration of PAMP, Fos expression is up-regulated in various areas of the hypothalamus (Ueta Y. et al.).

PAMP's antimicrobial activity is mediated by binding to bacterial molecules (Marutsuka K et al.).

### Physiological Activities

Numerous physiological activities are attributed to PAMP, such as activities relating to the control of fluid and electrolyte homeostasis, cardiac and neuronal activity, immune responses, and regulation of reproductive functions; in addition, PAMP has been shown to be a potent angiogenic factor.

PAMP is thought to be an autocrine regulator of adrenal steroid secretion. PAMP was found to cause a dose-dependent increase in release of steroids into the medium (Thomson L M et al.). However, PAMP concentration-dependently inhibits Ca2⁺-dependent, agonist-stimulated aldosterone and catecholamine secretion (Belloni A S et al; Nussdorfer G G.), and angiotensin II-stimulated, but not basal aldosterone secretion of dispersed human adrenocortical cells; in aldosteronoma dispersed cells, PAMP also depresses basal aldosterone secretion (Andreis P G et al.).

PAMP also has dose-dependent antimicrobial activity against E. coli in colonic mucosa, and plays a role in mucosal defense (Marutsuka K et al.).

### PAMP's Role in Hypotension and Vasodilation

PAMP causes a rapid and strong hypotensive effect in a dose-dependent manner upon intravenous injection (Kitamura K et al; Saita M et al.). PAMP plasma concentration is also significantly higher in hypertensive than normotensive subjects, and has a significant positive correlation with mean blood pressure and adrenomedullin concentration (Kuwasako K et al. 1999). Thus PAMP has potent hypotensive and vasodilatory effects.

PAMP's vasodilatory potency, though lower than that of AM, has also been described. Intradermal injection of PAMP induces transient vasodilatation, as measured using a Laser Doppler Imager, and causes itch sensation and local erythema. PAMP-induced histamine release is, however, not inhibited by Ca2⁺ (Nakamura M et al; Hasbak P et al.). On the other hand, AM-induced endothelium-dependent vasodilation can be converted to vasoconstriction in the presence of PAMP, probably through a nitric oxide-dependent pathway (Li J et al.).

PAMP may also have direct physiological effects on the heart based on its expression patters (see above), its cardiovascular and sympathetic effects, and its ability to affect and regulate hypotension (see above; Etoh T et al.). Furthermore, during the progression of chronic human heart failure (CHF), transcription of preproAM mRNA is upregulated in the lungs and in the failing ventricles, and an increase in the net release of the peptide is observed (Stangl K et al.).

### PAMP's Role in Neuronal Activity and Regulation

PAMP appears to act in the brain and pituitary gland to facilitate the loss of plasma volume, actions which complement its vasodilatory effects in blood vessels. Throughout the body, PAMP acts presynaptically to inhibit adrenergic nerves that innervate blood vessels (Samson W K; Shimosawa T et al. 1995).

PAMP acts as an antinicotinic peptide cosecreted with catecholamines by a Ca2⁺-dependent exocytosis in response to nicotinic receptor stimulation (Katoh F et al.). Furthermore, PAMP regulates catecholamine release and synthesis by interfering with nicotinic cholinergic receptors in chromaffin cells (Kobayashi H et al. 2001). The anticholinergic hypotensive actions of PAMP are due, at least in part, to PAMP-induced inhibition of catecholamine-synthesizing enzyme expression—an effect that is mediated by the cAMP/protein kinase A pathway (Takekoshi K et al.). Furthermore, PAMP inhibits norepinephrine release from peripheral sympathetic nerve endings (Shimosawa T.).

PAMP also causes physiological responses through the activation of a neural network in the hypothalamus and the brainstem (Ueta Y et al.).

PAMP inhibits the growth of neuroblastoma cells by inhibiting N-type Ca2⁺ channels through PTX-sensitive G protein-coupled receptors (see above)—a different mechanism/signaling pathway from AM-induced inhibition of cell growth (Ando K et al.).

### PAMPs Role in Reproductive Physiology

PAMP and follicle stimulating hormone (FSH) are co-stored in secretory granules in the pituitary gland, suggesting that PAMP, like FSH, is involved in the control of reproductive physiology (Montuenga L M et al.). Expression of PAMP in prostate carcinoma neuroendocrine cells increases upon androgen deprivation, suggesting that PAMP regulates androgen-independent prostate tumor growth (Calvo A et al.).

Furthermore, as discussed below, PAMP is a powerful angiogenic factor, and thereby may regulate a physiological process that is central for the female menstrual cycle.

### PAMP is a Potent Angiogenic Factor

Angiogenesis, the biological process leading to the generation of new blood vessels through sprouting or growth from pre-existing vessels, occurs and is essential for pre- and post-natal development, tissue growth, wound healing, and the cycle of the female reproductive system, and is a necessary factor in many pathological processes, such as the growth of solid tumors; it is also implicated in the pathophysiology of such diseases and conditions as arthritis, atherogenesis, corneal neovascularization, diabetic retinopathy, endometriosis, and psoriasis (Battegay E J.).

The process involves the migration and proliferation of endothelial cells from preexisting vessels.

The molecules responsible for regulating angiogenesic processes play an important role in tissue development and growth, wound healing (Rettura et al.) and the development of malignancies (Klagsbrun M et al; Brem S S et al.), and other diseases and conditions, and have thus been the subject of significant research efforts.

A variety of soluble mediators have been implicated in the induction of neovascularization. These include prostaglandins (Auerbach, 1981), human urokinase (Berman M et al.), copper (Raju K S et al.), and various “angiogenesis factors” (for instance, see U.S. Pat. No. 4,916,073).

A potent mitogen or growth factor present in many tissue types, basic fibroblast growth factor (bFGF), binds to extracellular matrix components, particularly heparin, from which it is released following injury. Once released, bFGF is a potent angiogenic factor.

Vascular endothelial growth factor (VEGF), a disulphide-bonded homodimeric, glycosylated protein of 46-48 kDa (24 kDa subunits), is a mitogen that appears to be highly specific for vascular endothelial cells, as it does not appear to enhance the proliferation of most other cell types. Potent synergy between VEGF and bFGF has been shown in the induction of angiogenesis and neo-vascularisation under physiological conditions. VEGF significantly influences vascular permeability and is a strong angiogenic protein in several bioassays. It has also been suggested that VEGF is released from smooth muscle cells and macrophages, and may play a role in the development of arteriosclerotic diseases.

PAMP functions as a potent angiogenic factor and in some assays has been shown to be even more potent than VEGF or bFGF (Martínez A et al. and US Patent, Application 20060160730). Because angiogenesis facilitates the growth, progression and metastasis of many types of cancer, PAMP is believed to play a role in increasing these deadly processes.

### SUMMARY OF THE INVENTION

Antibodies and antibody fragments that reduce or prevent binding of PAMP to PAMP receptors or other biological interacting partners of PAMP, and thereby interfere with the biological activities of PAMP, are disclosed.

In one embodiment, the invention provides an antibody, or antigen-binding fragment thereof that immunospecifically binds to a PAMP molecule comprising a sequence having at least 85% identity to the sequence of SEQ ID NO:4 (PAMP-20) or SEQ ID NO:6 (PAMP-12), or both.

Preferably, the antibody or antigen-binding fragment thereof binds to a PAMP molecule comprising a sequence having at least 35%, at least 40%, at least 45%, at least 50%, at least 55%, at least 60%, at least 65%, at least 70%, at least 75%, at least 80%, at least 85%, at least 90%, at least 95%, or at least 99% identity to SEQ ID NO:4 (PAMP-20) or SEQ ID NO:6 (PAMP-12), or both.

In another embodiment, the invention provides an antibody or antigen-binding fragment thereof that immunospecifically binds to a PAMP molecule comprising a sequence consisting of SEQ ID NO:4 (PAMP-20) or SEQ ID NO:6 (PAMP-12), or both.

In another embodiment, the invention provides a antibody, or antigen-binding fragment thereof that immunospecifically binds to a PAMP molecule encoded by a nucleotide sequence that hybrizes to the complement of SEQ ID NO:3 or SEQ ID NO:5.

In one embodiment according to the invention there is provided an anti-PAMP antibody generated by immunizing an animal with an epitope sequence contained within a peptide or polypeptide (hereinafter “(poly)peptide”) selected from the group consisting of
(i) a (poly)peptide comprising or consisting of an amino acid sequence of SEQ ID NO:4 (PAMP-20);
(ii) a (poly)peptide comprising or consisting of an amino acid sequence of SEQ ID NO:6 (PAMP-12);
(iii) a (poly)peptide comprising or consisting of an amino acid sequence of SEQ ID NO:2 (preproadrenomodullin);
(iv) a (poly)peptide encoded by a polynucleotide sequence that hybrizes to the complement of SEQ ID NO:1 (encoding preproadrenomodullin);
(v) a (poly)peptide encoded by a polynucleotide sequence that hybridizes to the complement of SEQ ID NO:3 (encoding PAMP-20); or
(vi) a (poly)peptide encoded by a polynucleotide sequence that hybridizes to the complement of SEQ ID NO:5 (encoding PAMP-12).

The stringency conditions for hybridization can be manipulated by a person skilled in the art of the present invention. Stringency conditions may be high or low stringency.

It will be appreciated by a person skilled in the art that the degree of hybridization of nucleotide sequences can be manipulated by varying certain parameters. For example, hybridization under stringent conditions may occur, e.g., by hybridization to filter-bound DNA in 6 times sodium chloride/sodium citrate (SSC) at about 45 degrees C. followed by one or more washes in 0.2 times SSC/0.1% SDS at about 50-65 degrees C.

Hybridization under highly stringent conditions may occur, e.g., by hybridization to filter-bound nucleic acid in 6 times SSC at about 45 degrees C. followed by one or more washes in 0.1 times SSC/0.2% SDS at about 68 degrees C. (see, for example, Ausubel F M et al.)

In another embodiment, the invention provides an antibody selected from the group consisting of:

(i) monoclonal antibody EGX-P-E9;
(ii) a chimeric antibody having heavy and light chain variable regions from EGX-P-E9 and human constant regions; or
(iii) a humanized antibody having complementarity determining regions (CDRs) from EGX-P-E9 and human variable domain framework and constant regions.

In another embodiment, the invention provides an antibody or antigen-binding fragment thereof comprising a heavy chain variable region sequence having at least 90% identity to SEQ ID NO: 11 (Heavy chain variable region of EGX-P-E9).

In another embodiment, the invention provides an antibody or antigen-binding fragment thereof comprising a light chain variable region sequence having at least 90% identity to SEQ ID NO:13 (Light chain variable region of EGX-P-E9).

Preferably, the antibody or antigen-binding fragment comprises a heavy or light chain variable region sequence having at least 35%, at least 40%, at least 45%, at least 50%, at least 55%, at least 60%, at least 65%, at least 70%, at least 75%, at least 80%, at least 85%, at least 90%, at least 95%, or at least 99% identity to SEQ ID NO:11 or SEQ ID NO:13 respectively.

In another embodiment, the invention provides an antibody or antigen-binding fragment thereof comprising a heavy chain variable region sequence having at least one complementarity determining region selected from the group consisting of DYYIH (SEQ ID NO: 14), YIDPENGETAYAPKFQG (SEQ ID NO:15), or PYFSLGRNY (SEQ ID NO:16).

Preferably, the heavy chain variable region comprises the sequences of DYYIH (SEQ ID NO: 14), YIDPENGETAYAPKFQG (SEQ ID NO:15), and PYFSLGRNY (SEQ ID NO:16).

In another embodiment, the invention provides an antibody or antigen-binding fragment thereof comprising a light chain variable region sequence having at least one complementarity determining region selected from the group consisting of RSSQSIVHGNGDTYLE (SEQ ID NO:17), KVSNRFS (SEQ ID NO:18) or FQGSHVPLT (SEQ ID NO:19).

Preferably, the light chain variable region comprises the sequences of RSSQSIVHGNGDTYLE (SEQ ID NO:17), KVSNRFS (SEQ ID NO:18) and FQGSHVPLT (SEQ ID NO:19).

Preferably the antibodies or antigen-binding fragments are selected from the group consisting of Fab, Fab′ and F(ab)₂, Fd, single-chain Fvs (scFv), single-chain antibodies, disulfide-linked Fvs (dsFv) and fragments comprising either a V_{L} or V_{H} domain.

In another embodiment, the invention provides an anti-PAMP antibody or antigen-binding fragment thereof which binds human, simian or murine PAMP-20.

Preferably, the antibody binds PAMP-20 with an affinity of at least 1000 nM, 200 nM, 50 nM, 10 nM, or 5 nM.

In another embodiment, the invention provides an antibody or antigen-binding fragment thereof which binds to an epitope to which monoclonal antibody EGX-P-E9 binds.

In another embodiment, the invention provides an antibody or antigen-binding fragment thereof that competitively inhibits binding of an antibody to an epitope recognised by the EGX-P-E9 monoclonal antibody.

In another embodiment, the invention provides an antibody or antigen-binding fragment thereof that competitively inhibits binding of antibody EGX-P-E9 to a PAMP sequence of the invention.

In a preferred embodiment, the antibody competitively inhibits binding to the epitope by at least 95%, at least 90%, at least 85%, at least 80%, at least 75%, at least 70%, at least 60%, or at least 50%.

In another embodiment, the invention provides an antibody or antigen-binding fragment thereof which binds to an epitope sequence present on PAMP, wherein the epitope comprises the sequence Trp Asn Lys Trp Ala Leu Ser Arg.

In another embodiment, the invention provides a humanised EGX-P-E9 antibody which has been affinity matured.

In another embodiment, the invention provides a humanised veneered EGX-P-E9 antibody.

In one embodiment, the antibody is a monoclonal. In another embodiment, the antibody is polyclonal. In another embodiment, polyclonal sera can be prepared in transgenic animal.

In another embodiment, the antibody is recombinant.

In another embodiment, the invention provides an antibody that inhibits or prevents activation of PAMP receptors or receptor complexes, or reduces, inhibits or prevents binding of PAMP-20 or PAMP-12 to PAMP receptors.

In another embodiment, the invention provides an anti-PAMP antibody, wherein by binding to PAMP, the antibody mediates one or more biological activites selected from the group consisting of:

(i) reducing, inhibiting or preventing activation of intracellular signalling;
(ii) reducing, inhibiting or preventing suppression of cellular Ca²⁺ influx;
(iii) reducing, inhibiting or preventing adenyl cyclase activation of cAMP production or release;
(iv) reducing, inhibiting or preventing up- or down regulation of gene expression (e.g. fos, catecholamine synthesizing enzymes);
(v) reducing, inhibiting or preventing suppression of the expression of pre-proadrenomedullin (ADM);
(vi) reducing, inhibiting or preventing suppression of the expression of genes encoding enzymes that synthesize catecholamine;
(vii) reducing, inhibiting or preventing suppression of the expression of tyrosine hydroxlase and dopamine β-hydroxylase;
(viii) reducing, inhibiting or preventing hormone secretion;
(ix) reducing, inhibiting or preventing suppression of AM or PAMP release;
(x) reducing, inhibiting or preventing histamine release;
(xi) reducing, inhibiting or preventing aldosterone or catecholamine release;
(xii) reducing, inhibiting or preventing release of steroid hormones;
(xiii) reducing, inhibiting or preventing release of DHEA;
(xiv) reducing, inhibiting or preventing a hypotensive reaction;
(xv) reducing, inhibiting or preventing a vasorelaxant reaction;
(xvi) reducing, inhibiting or preventing stimulation of cellular proliferation;
(xvii) reducing, inhibiting or preventing stimulation of microvascular endothelial cell proliferation;
(xviii) reducing, inhibiting or preventing angiogenesis;
(xix) reducing, inhibiting or preventing neuroblastoma cell proliferation; or
(xx) reducing, inhibiting or preventing cancer cell proliferation.

In another embodiment, the invention provides a peptide of less then 50 amino acids, preferably less than 30 amino acids, more preferably less than 20, still more preferably less than 10 amino acids, comprising the sequence Trp Asn Lys Trp Ala Leu Ser Arg. Preferably, the peptide contains a sequence of at least 4, at least 5, at least 6, at least 7, at least 8, at least 9, at least 10 amino acids.

In another embodiment, the invention provides a method of detecting and/or quantifying PAMP molecules, or fragments, variants, derivatives, conjugates, multimers or fusions thereof in a sample comprising:

(i) obtaining a sample from a subject;
(ii) contacting the sample with the an anti-PAMP antibody or antigen-binding fragment thereof of the invention;
(iii) detecting the binding of the anti-PAMP antibody or antigen-binding fragment thereof; and optionally
(iv) quantifying the level of PAMP in the sample.

Preferably, the sample is a blood sample, but could be any bodily fluid or tissue sample such as for example, plasma, lymphatic fluid, peritoneal fluid, cerebrospinal fluid, pleural fluid, synovial fluid.

In another embodiment, the invention provides a hybridoma cell line deposited to the ATCC and designated clone PTA-7958.

In another embodiment, the invention provides an antibody or antigen-binding fragment thereof that binds to a PAMP sequence of the invention with an off rate (k_{off}) of less than or equal to 5×10⁻² sec⁻¹, 10⁻² sec⁻¹, 5×10⁻³ sec⁻¹ or 10⁻³ sec 1.

More preferably, the antibody or antigen-binding fragment thereof binds to a PAMP sequence of the invention with an off rate (k_{off}) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹, or 10⁻⁷ sec 1.

In other embodiment, the invention provides an antibody or antigen-binding fragment thereof that binds to a PAMP sequence of the invention with an on rate (kₒₙ) of greater than or equal to 10³ M⁻¹ sec⁻¹, 5×10³ M⁻¹ sec⁻¹, 10⁴ M⁻¹ sec⁻¹ or 5×10⁴ M⁻¹ sec 1.

More preferably, the antibody or antigen-binding fragment thereof binds to a PAMP sequence of the invention with an on rate (kₒₙ) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹, or 10⁷ M⁻¹ sec⁻¹.

In one embodiment, the invention provides a pharmaceutical composition comprising an effective amount of an anti-PAMP antibody or antigen-binding fragment thereof according to the invention together with a pharmaceutically acceptable carrier.

In another embodiment, the invention provides a method of treating a PAMP-responsive condition, disorder, or disease in a subject, comprising administering to the subject a pharmaceutical composition according to the invention.

Methods of treating a subject for a condition, disorder, or disease that is either as result of a disturbance in PAMP expression levels, or that can be corrected or treated by administration of an antibody or fragment thereof of the present invention are provided; such conditions, disorders, or diseases include, but are not limited to, hypertension, neurological disorders, cancers, inflammatory disorders such as arthritis, pathologies resulting from or exacerbated by excessive angiogenesis, and microbial infections.

In another embodiment, the invention provides a method of treating angiogenesis in a subject, comprising administering to the subject a pharmaceutical composition according to the invention.

In another embodiment, the invention provides a method of treating a pathological condition that is exacerbated by angiogenesis comprising administering to the subject a pharmaceutical composition according to the invention.

Preferably, the pathological condition that is exacerbated by angiogenesis is selected from the group consisting of cancer, macular degeneration, diabetic blindness, rheumatoid arthritis, psoriasis, coronary artery disease and stroke.

In another embodiment, the invention provides a method of diagnosing a PAMP-responsive condition, disorder or disease in a subject, comprising:

(i) obtaining a sample from a subject;
(ii) contacting the sample with the an anti-PAMP antibody or antigen-binding fragment thereof of the invention;
(iii) measuring the expression of PAMP;
wherein an increase in PAMP expression relative to a control sample is indicative of a condition, disorder or disease.

In another embodiment, the invention provides a method of diagnosing a PAMP-responsive condition in a subject, the method comprising contacting a sample from a subject with an antibody, or antigen-binding fragment thereof of the present invention for a time and under conditions sufficient for the antibody or antigen-binding fragment thereof to bind PAMP and form an antigen-antibody complex and detecting the complex wherein an enhanced level of the complex compared to a control sample indicates that the subject suffers from a PAMP responsive condition.

In one embodiment, an increase in PAMP expression levels is indicative of a reproductive disorder.

In one embodiment, diagnosing a PAMP responsive condition is performed in vivo, e.g., by imaging.

In another embodiment, the invention provides methods of identifying or isolating additional ligands, antibodies or other sustances which bind PAMP, including inhibitors and/or promoters of PAMP binding to its receptor.

The present invention also provides a nucleic acid encoding an anti-PAMP antibody or antigen-binding fragment thereof as described herein according to any embodiment.

The present invention also provides a cell expressing an anti-PAMP antibody or antigen-binding fragment thereof as described herein according to any embodiment, e.g., a hybridoma or a transfectoma.

The present invention also provides a method for producing an anti-PAMP antibody or antigen-binding fragment of the present invention, said method comprising culturing a cell of the present invention for a time and under conditions for said antibody to be expressed. Preferably, the method additionally comprises isolating the antibody or antigen-binding fragment.

The present invention also provides for use of an antibody and/or antigen-binding fragment thereof or a composition as described herein according to any embodiment in medicine.

In another embodiment, the invention provides for the use of an anti-PAMP antibody or antigen-binding fragment thereof in the manufacture of a medicament for treating a PAMP-responsive condition, disorder, or disease in a subject.

In another embodiment, invention also provides therapeutic/diagnostic kits comprising anti-PAMP antibodies, and/or antigen-binding fragments thereof of the present invention for use in the present methods.

Preferably, the subject according to the methods of the invention is a human subject.

### BRIEF DESCRIPTION OF THE DRAWINGS

FIG. 1. shows an ELISA data measuring the affinity of the PAMP antibody EGX-P-E9 for human PAMP.

FIG. 2. shows competitive ELISA results for the antibody EGX-P-E9.

FIG. 3. shows the anti-angiogenic effects of antibody EGX-P-E9 in a matrigel plug angiogenesis assay in mice.

FIG. 4A shows the nucleotide and amino acid sequences of the heavy chain variable region of mouse monoclonal antibody E9. The leader sequence and the complementarity determining regions (CDRs) are indicated by an underline and boxes. The CDRs were identified according to Kabat definition.

FIG. 4B shows the nucleotide and amino acid sequences of the light chain variable region of mouse monoclonal antibody E9. The leader sequence and the complementarity determining regions (CDRs) are indicated by an underline and boxes. The CDRs are identified according to Kabat definition.

### KEY TO SEQUENCE LISTING

SEQ ID NO:1—Preproadrenomudullin ORF nucleotide sequence.
SEQ ID NO:2—Preproadrenomudullin amino acid sequence.
SEQ ID NO:3—Human PAMP-20 nucleotide sequence.
SEQ ID NO:4—Human PAMP-20 amino acid sequence.
SEQ ID NO:5—Human PAMP-12 nucleotide sequence.
SEQ ID NO:6—Human PAMP-12 amino acid sequence.
SEQ ID NO:7—Preproadrenomudullin cDNA nucleotide sequence of NCBI sequence database (Genbank Accession No. E09705).
SEQ ID NO:8—Rat PAMP amino acid sequence.
SEQ ID NO:9—Mouse PAMP amino acid sequence.
SEQ ID NO:10-Nucleotide sequence of heavy chain variable region of antibody EGX-P-E9.
SEQ ID NO:11—Amino acid sequence of heavy chain variable region of antibody EGX-P-E9.
SEQ ID NO:12—Nucleotide sequence of light chain variable region of antibody EGX-P-E9.
SEQ ID NO:13—Amino acid sequence of light chain variable region of antibody EGX-P-E9.
SEQ ID NO:14—Amino acid sequence of VHCDR1.
SEQ ID NO:15—Amino acid sequence of VHCDR2.
SEQ ID NO:16—Amino acid sequence of VHCDR3.
SEQ ID NO:17—Amino acid sequence of VLCDR1.
SEQ ID NO:18—Amino acid sequence of VLCDR2.
SEQ ID NO:19—Amino acid sequence of VLCDR3.
SEQ ID NO:20—synthetic primer.
SEQ ID NO:21—synthetic oligonucleotide.
SEQ ID NO:22—synthetic oligonucleotide.
SEQ ID NO:23—synthetic oligonucletoide.
SEQ ID NO:24—synthetic primer.
SEQ ID NO:25—Amino acid sequence of human PAMP (8-20).
SEQ ID NO:26—Amino acid sequence of human PAMP (10-20).
SEQ ID NO:27—Amino acid sequence of human PAMP (12-20).
SEQ ID NO:28—Amino acid sequence of human PAMP (13-20).

### DETAILED DESCRIPTION OF THE INVENTION

In light of the diverse biological activities described above, it is not surprising that PAMP is implicated in a number of physiological and disease conditions, including, but not limited to, cancer, wound healing, hypotension, and neurological indications, and in the regulation of other medically relevant functions, such as reproductive physiology. Disturbances in the regulation of PAMP's activity may be involved in such diseases, disorders, and/or conditions. Alternatively, modulation of PAMP activity, where its own activity is well regulated, may serve to off-set disturbances in the regulation of other systems in the organism which may be involved in certain other diseases, disorders, and/or conditions, such as, for example, but not limited to, cancerous growth(s). Therefore, there is a need for the identification, characterization, and development of agents that modulate PAMP activity, such as the antibodies of the present invention, which can play a role in preventing, ameliorating or correcting such diseases, disorders, and/or conditions.

The present inventors have demonstrated that an antibody raised against a PAMP epitope conserved across mouse, rat and human is able to inhibit PAMP-mediated physiological activities and effects.

### Epitopes and Antigens

By “epitope” is intended the part of an antigenic molecule to which an antibody is produced and to which the antibody will bind. The term “epitope,” as used herein, refers to (a) portion(s) of a peptide having antigenic or immunogenic activity in an animal, preferably a vertebrate, more preferably a mammal, and most preferably in a human or a transgenic animal expressing relevant components of the human immune system. Epitopes may comprise proteins, protein fragments, peptides, carbohydrates, lipids, and other molecules, but for the purposes of the present invention are most commonly short oligopeptides.

The term “epitope” is intended to encompass an “immunogenic epitope”, an “antigenic epitope”, or “antigen epitope”.

Additional non-exclusive preferred antigenic epitopes include the antigenic epitopes disclosed herein, as well as portions thereof.

The epitopes of the invention are preferably contained within the sequence of SEQ ID NO:4 (PAMP-20) or SEQ ID NO:6 (PAMP-12).

Epitopes can be used as the target molecules in immunoassays. (See, for instance, Wilson I A et al; Sutcliffe J G et al.).

Furthermore, epitope bearing peptides of the invention may be modified, for example, by the addition of amino acids to the peptides, for example, but not limited to, at the amino- and/or carboxy-termini of the peptide.

Other modifications of epitope-bearing peptides contemplated by this invention include biotinylation.

Similarly, immunogenic epitopes can be used, for example, to induce antibodies according to methods known in the art. (See, for example, Wilson I A et al. and Sutcliffe J G et al.). Preferred immunogenic epitopes include the immunogenic epitopes disclosed herein. The peptides comprising one or more immunogenic epitopes may be presented for eliciting an antibody response together with a carrier protein, such as an albumin, to an animal system (such as rabbit or mouse). However, immunogenic epitopes comprising as few as 8 to 10 amino acids have been shown to be sufficient to raise antibodies capable of binding to epitopes.

The antigen peptide may be coupled to a macromolecular carrier, such as, for example, but not limited to, keyhole limpet hemacyanin (KLH) or tetanus toxoid. For instance, peptides containing cysteine residues, and that are expressed or synthesized to contain cysteine, for example, but not limited to, at the N- and C-termini, may be coupled to a carrier using a linker such as, but not limited to, maleimidobenzoyl-N-hydroxysuccinimide ester (MBS), while other peptides may be coupled to carriers using a more general linking agent such as glutaraldehyde.

Epitope bearing peptides of the invention may also be synthesized as multiple antigen peptides (MAPs) with or without T cell epitopes. MAPs consist of multiple copies of a specific peptide attached to a non-immunogenic lysine core. Map peptides usually contain four or eight copies of the peptide often referred to as MAP-4 or MAP-8 peptides. By way of non-limiting example, MAPs may be synthesized onto a lysine core matrix attached to a polyethylene glycol-polystyrene (PEG-PS) support. The peptide of interest is synthesized onto the lysine residues using 9-fluorenylmethoxycarbonyl (Fmoc) chemistry. For example, MAP resins, such as, for example, the Fmoc Resin 4 Branch and the Fmoc Resin 8 Branch which can be used to synthesize MAPs, are commercially available. Cleavage of MAPs from the resin may be performed with standard trifloroacetic acid (TFA)-based cocktails known in the art. MAP peptides may be used as an immunizing vaccine which elicits antibodies that recognize both the MAP and the native protein from which the peptide was derived.

An epitope may also be fused to other polypeptide sequences. For example, the peptides of the present invention may be fused with the constant domain of immunoglobulins (IgA, IgE, IgG, IgM), or portions thereof (CH1, CH2, CH3, or any combination thereof and portions thereof) or, as non-limiting examples, albumin and transferin (including but not limited to recombinant human albumin or fragments or variants thereof, see, e.g., U.S. Pat. No. 5,876,969; EP Patent 0413622; U.S. Pat. No. 5,766,883; and U.S. Pat. No. 7,176,278), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker A et al. Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al. Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht R et al. 1991).

Antigens may also be derivatives in that they are modified, i.e., by the covalent attachment of any type of molecule to the antigen. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Any of numerous methods of cleavage may be applied, including cleavage by cyanogen bromide, trypsin, chymotrypsin, papain, V8 protease, NaBH₄, acetylation, formylation, oxidation, reduction, metabolic synthesis in the presence of tunicamycin, etc.

In addition, antigenic molecules of the invention may be chemically synthesized. For example, a peptide corresponding to a portion of a protein can be synthesized by use of a peptide synthesizer. Furthermore, if desired, non-classical amino acids or chemical amino acid analogs can be introduced as substitutions and/or additions into the sequence of one, any, both, several or all of the peptides of the complex.

Non-classical amino acids include, but are not limited to, the D-isomers of the common amino acids, fluoro-amino acids, designer amino acids such as beta-methyl amino acids, C gamma-methyl amino acids, N gamma-methyl amino acids, and amino acid analogs in general.

Examples of non-classical amino acids include: alpha-aminocaprylic acid, Acpa; (S)-2-aminoethyl-L-cysteine HCl, Aecys; aminophenylacetate, Afa; 6-amino hexanoic acid, Ahx; gamma-amino isobutyric acid and alpha-aminoisobytyric acid, Aiba; alloisoleucine, Aile; L-allylglycine, Alg; 2-amino butyric acid, 4-aminobutyric acid, and alpha-aminobutyric acid, Aba; p-aminophenylalanine, Aphe; b-alanine, Bal; p-bromophenylalaine, Brphe; cyclohexylalanine, Cha; citrulline, Cit; beta-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-fluorophenylalanine, Fphe; D-glucoseaminic acid, Gaa; homoarginine, Hag; delta-hydroxylysine HCl, Hlys; DL-beta-hydroxynorvaline, Hnyl; homoglutamine, Hog; homophenylalanine, Hoph; homoserine, Hos; hydroxyproline, Hpr; p-iodophenylalanine, Iphe; isoserine, Ise; alpha-methylleucine, Mle; DL-methionine-5-methylsulfoniumchloide, Msmet; 3-(1-naphthyl)alanine, 1Nala; 3-(2-naphthyl)alanine, 2Nala; norleucine, Nle; N-methylalanine, Nmala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, On-Ayr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluoroalanine, Tfa; 6-hydroxydopa, Thphe; L-vinylglycine, Vig;

(−)-(2R)-2-amino-3-(2-aminoethylsulfonyl)propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl)propanoic acid, Ahsopa; (−)-(2R)-2-amino-3-(2-hydroxyethylsulfanyl)propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda; (S)-5,5-difluoronorleucine, Dfnl; (S)-4,4-difluoronorvaline, Dfnv; (3R)-1-1-dioxo-[1,4]thiaziane-3-carboxylic acid, Dtca; (S)-4,4,5,5,6,6,6-heptafluoronorleucine, Hfnl; (S)-5,5,6,6,6-pentafluoronorleucine, Pfnl; (S)-4,4,5,5,5-pentafluoronorvaline, Pfnv; and (3R)-1,4-thiazinane-3-carboxylic acid, Tea. Furthermore, the amino acid can be D (dextrorotary) or L (levorotary). For a review of classical and non-classical amino acids, see Sandberg et al.

### Production of Antigen

Polypeptides, fragments, variants, derivates, multimers, conjugates, and fusion proteins of the sequences of the invention may be used as antigens of the present invention.

Polypeptides, fragments, variants, derivates, multimers, conjugates, and fusion proteins of the above sequences, which function as epitopes, may be synthesized or produced by any conventional means. (See, e.g., Houghten R A., further described in U.S. Pat. No. 4,631,211).

In one embodiment of the invention, the antigen is expressed recombinantly from a nucleotide sequence encoding the amino acid sequence of a polypeptide antigen in prokaryotic or eukaryotic expression systems, such as, for example, but not limited to, E. coli, yeast, insect, such as, for example Sf9 cells infected by an antigen-specific baculovirus (expression vector) or drosophila cell lines, murine, such as, for example, Chinese Hamster Ovary (CHO) cells, simian, such as, for example, COS cells, human cells lines, such as, for example, HeLa cells, or any other system for recombinant production of protein.

For example, epitope bearing peptides of the invention may be expressed in baculovirus infected insect cells, such as Sf9 cells, whereby such cells may be used as the immunogen. Production of the Sf 9 (Spodoptera frugiperda) cells is disclosed in U.S. Pat. No. 6,004,552.

### Immunization Methods

Epitope-bearing peptides or polypeptides of the present invention may be used to induce antibodies according to methods known in the art including, but not limited to, in vivo immunization, in vitro immunization, and phage display methods. See, for example, Wilson I A et al and Sutcliffe J G et al.

For in vivo immunizations, animals such as rabbits, rats and mice are immunized with either free or carrier-coupled peptides or MAP peptides of emulsions containing an effective amount of peptide or carrier protein, often an amount between 50 and 200 micrograms/injection; the epitope-bearing peptide, free or carrier-coupled, is preferably emulsified in Freund's adjuvant or any other adjuvant known for stimulating an immune response. Immunization can also be performed by mixing or emulsifying the antigen-containing solution in saline, preferably in an adjuvant such as Freund's complete adjuvant, and injecting the mixture or emulsion parenterally, generally subcutaneously, intramuscularly, intraperitoneally and/or intradermally, though other routes may be effective, as well. One or several booster injections of the above antigen, for example, but not limited to, in saline, and preferably using an adjuvant, such as, but not limited to, Freund's incomplete adjuvant, may be useful or needed, for instance, at intervals of effective periods of time, often about two weeks, to provide a useful titer of antibody which can be detected, for example, by ELISA assay using biotinylated peptide (PAMP or a fragment thereof) attached to an avidin- or streptavidin-coated surface.

Polyclonal antisera are obtained by bleeding the immunized animal into a container, incubating the blood at 25 degrees C. for one hour, followed by incubating at 4 degrees C. for 2-18 hours. The serum is recovered by centrifugation (e.g., 1,000 g for 10 minutes). About 20-50 ml per bleed may be obtained from rabbits. The titer of antibodies in serum from an immunized animal may be increased by selection of antigen-specific antibodies, for instance, by adsorption to the peptide on a solid support and elution of the selected antibodies according to methods known to a person of ordinary skill in the art.

One may alternatively generate antibodies by in vitro immunization using methods known in the art, preferably for the production of monoclonal antibodies, which for the purposes of this invention is considered equivalent to in vivo immunization.

### Methods of Isolating Monoclonal Antibodies

Any methods known to one of ordinary skill in the art may be used to identify and/or isolated cells expressing antibodies of the present invention. For example, after immunization of the animal, the spleen (and optionally, several large lymph nodes) are removed and dissociated into single cells. The spleen cells may be screened by applying a cell suspension to a plate or well coated with the antigen of interest. The B cells expressing membrane-bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen of interest (and that do not bind to unrelated antigens, see below). The selected monoclonal antibody (mAb)-secreting hybridomas are then cultured either in vitro (e.g., in tissue culture bottles or hollow fiber reactors), or in vivo (as ascites in mice).

Antibodies or antibody fragments can also be isolated from antibody phage libraries generated using the techniques described in, for example, McCafferty et al; U.S. Pat. No. 5,514,548; and Marks et al. 1991 describe the isolation of murine and human antibodies, respectively, using phage libraries.

Subsequent publications describe the production of high affinity (nM range) human antibodies by chain shuffling (Marks J D et al.), as well as combinatorial infection and in vivo recombination as a strategy for constructing very large phage libraries (Waterhouse P et al.).

In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide-stabilized Fv antibody domains recombinantly fused to either the phage gene III or gene VIII protein. Thus, these techniques are viable alternatives to traditional monoclonal antibody hybridoma techniques for isolation of monoclonal antibodies.

Other examples of phage display methods that can be used to make the antibodies of the present invention include those disclosed in Brinkmann U et al; Kettleborough C A et al; Burton D R & Barbas C F; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Another method for producing monoclonal human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds., 1994, John Wiley & Sons, NY. The source of B cells for transformation is commonly human peripheral blood, but B cells for transformation may also be derived from other sources including, but not limited to, lymph nodes, tonsil, spleen, tumor tissue, and infected tissues. Tissues are generally made into single cell suspensions prior to EBV transformation. Additionally, steps may be taken to either physically remove or inactivate T cells (e.g., by treatment with cyclosporin A) in B cell-containing samples, because T cells from individuals seropositive for anti-EBV antibodies can suppress B cell immortalization by EBV. In general, the sample containing human B cells is inoculated with EBV, and cultured for 3-4 weeks. A typical source of EBV is the culture supernatant of the B95-8 cell line (ATCC #VR-1492). Initially, EBV lines are generally polyclonal. However, over prolonged periods of cell cultures, EBV lines may become monoclonal as a result of the selective outgrowth of particular B cell clones. Alternatively, polyclonal EBV transformed lines may be subcloned (e.g., by limiting dilution culture) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human×mouse; e.g., SPAM-8, SBC-H20, and CB-F7), and human cell lines (e.g., GM 1500, SKO-007, RPMI 8226, and KR-4). Thus, the present invention also provides a method of generating human antibodies against peptides of the invention or fragments thereof, comprising EBV-transformation of human B cells.

### Antibodies of the Invention

The anti-PAMP antibodies of the instant invention include polyclonal, monoclonal and recombinant antibodies. The antibodies bind PAMP molecules, or fragments, variants, derivatives, conjugates, multimers, or fusions thereof, and thereby inhibit activation of PAMP receptors, downstream signaling, and activation and suppression of gene expression and wider physiological effects mediated by PAMP binding to a receptor and thereby causing its activation in the absence of such antibodies.

An example of such an anti-PAMP antibody is the murine monoclonal antibody designated herein as EGX-P-E9, which can be produced recombinately, or by any method known in the art. The monoclonal antibody is produced by the hybridoma cell line deposited to the ATCC and designated clone PTA-7958. This monoclonal antibody binds an epitope within the C-terminal 8 amino acids of the PAMP molecule, which is a highly conserved sequence.

The term “antibody,” as used herein, refers to immunoglobulin molecules and immunologically active portions or fragments of immunoglobulin molecules, including T cell receptor molecules, i.e., molecules that contain an antigen binding site that immunospecifically binds an antigen. As such the term “antibody” encompasses not only whole antibody molecules, but also antibody multimers and antibody fragments and/or variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a V_{L} or a V_{H} domain. The term “single chain Fv” or “scFv” as used herein refers to a polypeptide comprising a V_{L} domain of antibody linked to a V_{H} domain of an antibody.

Antibodies of the invention include, but are not limited to, recombinant, monoclonal, multispecific, human, humanized or chimeric antibodies, veneered antibodies, single chain antibodies, Fab fragments, F(ab′) fragments, fragments produced by a Fab expression library, anti-idiotypic (anti-Id) antibodies (including, e.g., anti-Id antibodies to antibodies against a receptor molecule of the antigen of the present invention), intracellularly-made antibodies (i.e., intrabodies), and epitope-binding fragments of any of the above.

The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In a preferred embodiment, the immunoglobulin is an IgM isotype. In another preferred embodiment, the immunoglobulin is an IgG1 isotype. In another preferred embodiment, the immunoglobulin is an IgG2 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms.

By “isolated antibody” is intended an antibody removed from its native environment. Thus, an antibody produced by, purified from and/or contained within a hybridoma and/or a recombinant host cell is considered isolated for purposes of the present invention.

Binding site chains of antibodies all exhibit the same general structure of relatively conserved framework regions (FR) joined by three hyper variable regions, also known as complementarity determining regions or CDRs. From N-terminal to C-terminal, both heavy and light chain variable regions comprise the domains FR1, CDR1, FR2, CDR2, FR3, CDR3 and FR4. The assignment of amino acids to each domain is often in accordance with the definitions of Kabat Sequences of Proteins of Immunological Interest (National Institutes of Health, Bethesda, Md., 1987 and 1991), or Chothia C & Lesk; Chothia C et al.).

The present inventors have determined the complementarity determining regions of monoclonal antibody EGX-P-E9. The sequence of the complementarity determining regions are as follows: Variable heavy chain CDR1 (DYYIH; SEQ ID NO:14), CDR2 (YIDPENGETAYAPKFQG; SEQ ID NO:15), CDR3 (PYFSLGRNY; SEQ ID NO:16), and variable light chain CDR1 (RSSQSIVHGNGDTYLE; SEQ ID NO:17), CDR2 (KVSNRFS; SEQ ID NO:18) and CDR3 (FQGSHVPLT; SEQ ID NO:19).

By “Fab” is intended a monovalent antigen-binding fragment of an immunoglobulin that is composed of the light chain and part of the heavy chain.

By F(ab′)₂ is intended a bivalent antigen-binding fragment of an immunoglobulin that contains both light chains and part of both heavy chains.

By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778, 5,260,203, 5,455,030, and 5,856,456. Generally, the Fv polypeptide further comprises a polypeptide linker between the V_{H} and V_{L} domains that enables the sFv to form the desired structure for antigen-binding. For a review of sFv see Pluckthun (1994). The V_{H} and V_{L} domain complex of Fv fragments may also be stabilized by a disulfide bond (U.S. Pat. No. 5,747,654).

A bispecific or bifunctional antibody is a hybrid antibody having two different heavy/light chain pairs and two different binding sites. Bispecific antibodies can be produced by a variety of methods including fusion of hybridomas or linking of Fab′ fragments. See, for example, Songsivilai S & Lachmann P G; Kostelny S A et al. In addition, bispecific antibodies may be formed as “diabodies” (Holliger P et al.) or “janusins” (see Traunecker A et al. 1991.).

In another embodiment, the antibodies of the invention may also include multimeric forms of antibodies. For example, antibodies of the invention may take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known to one of ordinary skill in the art. For example, some percentage of purified antibody preparations (e.g., purified IgG1 molecules) spontaneously form protein aggregates containing antibody homodimers, and other higher-order antibody multimers. Alternatively, antibody homodimers may be formed through chemical linkage techniques known in the art. As a non-limiting example, heterobifunctional crosslinking agents including, but not limited to, SMCC [succinimidyl 4-(maleimidomethyl)cyclohexane-1-carboxylate] and SATA [N-succinimidyl S-acethylthio-acetate] (available, for example, from Pierce Biotechnology, Inc. (Rockford, Ill.)) can be used to form antibody multimers. An exemplary protocol for the formation of antibody homodimers is given in Ghetie M A et al. Antibody homodimers can be converted to F(ab)₂ homodimers through digestion with pepsin. Another way to form antibody homodimers is through the use of the autophilic T15 peptide described in Zhao Y & Kohler H.

Alternatively, antibodies can be made to multimerize naturally or through recombinant DNA techniques. IgM and IgA naturally form antibody multimers through the interaction with the J chain polypeptide. Non-IgA or non-IgM molecules, such as IgG molecules, can be engineered to contain the J chain interaction domain of IgA or 1μM, thereby conferring the ability to form higher order multimers on the non-IgA or non-IgM molecules (see for eample, Chintalacharuvu K R et al.) ScFv dimmers can also be formed through recombinant techniques known in the art; and example of the construction of scFv dimers is given in Goel A et al. Antibody multimers may be purified by any suitable method known in the art, e.g. size exclusion chromatography.

Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains.

“Specific binding” or “immunospecific binding” by an antibody means that the antibody binds (a) specific antigen molecule(s), or fragments, variants, or derivates, multimers, or fusion proteins thereof, but does not significantly bind to (i.e., cross react with) antigens, such as, for example, other structurally or functionally related proteins, or proteins with sequence homology. An antibody that binds the antigen of this invention and does not cross-react with other proteins is not necessarily an antibody that does not bind said other proteins under any or all conditions; rather, the antigen-specific antibody of the invention preferentially binds the antigen compared to its ability to bind said other antigens such that it will be suitable for use in at least one type of treatment, i.e. result in no unreasonable adverse effects in treatment.

In another embodiment, the antibodies of the present invention may be monospecific, bispecific, trispecific or of greater multispecificity. Multispecific antibodies may be specific for different epitopes of a peptide of the present invention or may be specific for both a peptide of the present invention as well as for a heterologous epitope, such as a heterologous polypeptide. See, e.g., PCT publications WO 93/17715; WO 92/08802; WO 91/00360; WO 92/05793; Tutt A et al; U.S. Pat. Nos. 4,474,893; 4,714,681; 4,925,648; 5,573,920; 5,601,819; Kostelny S A et al.

Preferably, the antibodies of the invention bind immunospecifically to human and/or monkey PAMP peptides.

Preferably, the antibodies of the invention bind immunospecifically to human and/or murine PAMP peptides.

Given that antigen-specific antibodies bind to epitopes of the antigen, an antibody that specifically binds antigen may or may not bind fragments of the antigen and/or variants of the antigen (e.g., peptides that are at least 95% identical to the antigen) depending on the presence or absence of the epitope bound by a given antigen-specific antibody in the antigen fragment or variant. Likewise, antigen-specific antibodies of the invention may bind species orthologues of the antigen (including fragments thereof) depending on the presence or absence of the epitope recognized by the antibody in the orthologue. Additionally, antigen-specific antibodies of the invention may bind modified forms of the antigen, for example, antigen fusion proteins. In such a case when antibodies of the invention bind the antigen fusion proteins, the antibody must make binding contact with the antigen moiety of the fusion protein in order for the binding to be specific for the antigen. Antibodies that specifically bind the antigen can be identified, for example, by immunoassays or other techniques known to those of skill in the art.

By way of non-limiting example, an antibody may be considered to bind a first antigen preferentially if it binds said first antigen with a dissociation constant (K_{D}) that is less than the antibody's K_{D} for the second antigen. In another non-limiting embodiment, an antibody may be considered to bind a first antigen preferentially if it binds said first antigen with an affinity that is at least one order of magnitude less than the antibody's K_{D} for the second antigen. In another non-limiting embodiment, an antibody may be considered to bind a first antigen preferentially if it binds said first antigen with an affinity that is at least two orders of magnitude less than the antibody's K_{D} for the second antigen.

In another non-limiting embodiment, an antibody may be considered to bind a first antigen preferentially if it binds said first antigen with an off rate (k_{off}) that is less than the antibody's k_{off} for the second antigen. In another non-limiting embodiment, an antibody may be considered to bind a first antigen preferentially if it binds said first antigen with an affinity that is at least one order of magnitude less than the antibody's k_{off} for the second antigen. In another non-limiting embodiment, an antibody may be considered to bind a first antigen preferentially if it binds said first antigen with an affinity that is at least two orders of magnitude less than the antibody's k_{off} for the second antigen.

Antibodies of the present invention may also be described or specified in terms of their binding affinity to vertebrate PAMP-20 peptides, or fragments, variants, or derivatives thereof. Preferred binding affinities include those with a dissociation constant or K_{D} less than 5×10⁻²M, 10⁻²M, 5×10⁻³M, 10⁻³M, 5×10⁻⁴M, 10⁻⁴M. More preferred binding affinities include those with a dissociation constant or K_{D} less than 5×10⁻⁵M, 10⁻⁵M, 5×10⁻⁶M, 10⁻⁶M, 5×10⁻⁷M, 10⁻⁷M, 5×10⁻⁸M or 10⁻⁸M. Even more preferred binding affinities include those with a dissociation constant or K_{D} less than 5×10⁻⁹M, 10⁻⁹M, 5×10⁻¹⁰M, 10⁻¹⁰M, 5×10⁻¹¹M, 10⁻¹¹M, 5×10⁻¹² M, 10⁻¹²M, 5×10⁻¹³M, 10⁻¹³M, 5×10⁻¹⁴M, 10⁻¹⁴M, 5×10⁻¹⁵M, or 10⁻¹⁵M.

In one embodiment of the present invention, antibodies that immunospecifically bind antigen peptides of the invention, comprise a polypeptide having the amino acid sequence of a heavy chain of an antibody of the invention and/or a light chain of an antibody of the invention.

In another embodiment of the present invention, antibodies that immunospecifically bind antigen peptides of the invention, comprise a polypeptide having the amino acid sequence of a V_{H} domain of a heavy chain of an antibody of the invention and/or a V_{L} domain of a light chain of an antibody of the invention.

In preferred embodiments, antibodies of the present invention comprise the amino acid sequence of a V_{H} domain and V_{L} domain of an antibody of the invention. Molecules comprising, or alternatively consisting of, antibody fragments or variants of the V_{H} and/or V_{L} domains of an antibody of the invention that immunospecifically bind the antigen of the invention are also encompassed by the invention, as are nucleic acid molecules encoding these V_{H} and V_{L} domains, molecules, fragments and/or variants.

The present invention also provides antibodies that immunospecifically bind antigen peptides of the invention, wherein said antibodies comprise, or alternatively consist of, a polypeptide having an amino acid sequence of any one, two, or three, of the V_{H} CDRs contained in a heavy chain of an antibody of the invention. In particular, the invention provides antibodies that immunospecifically bind antigen peptides of the invention, comprising, or alternatively consisting of, a polypeptide having the amino acid sequence of a V_{H} CDR1 contained in a heavy chain of an antibody of the invention. In another embodiment, antibodies that immunospecifically bind antigen peptides of the invention, comprise, or alternatively consist of, a polypeptide having the amino acid sequence of a V_{H} CDR2 contained in a heavy chain of an antibody of the invention. In a preferred embodiment, antibodies that immunospecifically bind antigen peptides of the invention, comprise, or alternatively consist of a polypeptide having the amino acid sequence of a V_{H} CDR3 contained in a heavy chain of an antibody of the invention. Molecules comprising, or alternatively consisting of, these antibodies, or antibody fragments or variants thereof, that immunospecifically bind antigen peptides of the invention are also encompassed by the invention, as are nucleic acid molecules encoding these antibodies, molecules, fragments and/or variants.

The present invention also provides antibodies that immunospecifically bind antigen peptides of the invention, wherein said antibodies comprise, or alternatively consist of, a polypeptide having an amino acid sequence of any one, two, or three of the V_{L} CDRs contained in a light chain of an antibody of the invention. In particular, the invention provides antibodies that immunospecifically bind antigen peptides of the invention, comprising, or alternatively consisting of, a polypeptide having the amino acid sequence of a V_{L} CDR1 contained in a light chain of an antibody of the invention. In another embodiment, antibodies that immunospecifically bind antigen peptides of the invention, comprise, or alternatively consist of, a polypeptide having the amino acid sequence of a V_{L} CDR2 contained in a light chain of an antibody of the invention. In a preferred embodiment, antibodies that immunospecifically bind antigen peptides of the invention, comprise, or alternatively consist of a polypeptide having the amino acid sequence of a V_{L} CDR3 contained in a light chain of an antibody of the invention. Molecules comprising, or alternatively consisting of, these antibodies, or antibody fragments or variants thereof, that immunospecifically bind antigen peptides of the invention are also encompassed by the invention, as are nucleic acid molecules encoding these antibodies, molecules, fragments and/or variants.

The present invention also provides antibodies (including molecules comprising, or alternatively consisting of, antibody fragments or variants) that immunospecifically bind antigen peptides of the invention, wherein said antibodies comprise, or alternatively consist of, one, two, or three, V_{H} CDRs and one, two, or three V_{L} CDRs, as contained in a heavy chain or light chain of an antibody of the invention. In particular, the invention provides for antibodies that immunospecifically bind antigen peptides of the invention, wherein said antibodies comprise, or alternatively consist of, a V_{H} CDR1 and a V_{L} CDR1, a V_{H} CDR1 and a V_{L} CDR2, a V_{H} CDR1 and a V_{L} CDR3, a V_{H} CDR2 and a V_{L} CDR1, V_{H} CDR2 and V_{L} CDR2, a V_{H} CDR2 and a V_{L} CDR3, a V_{H} CDR3 and a V_{H} CDR1, a V_{H} CDR3 and a V_{I}, CDR2, a V_{H} CDR3 and a V_{L} CDR3, or any combination thereof, of the V_{H} CDRs and V_{L} CDRs contained in a heavy chain or light chain of an antibody of the invention. Molecules comprising, or alternatively consisting of, fragments or variants of these antibodies, that immunospecifically bind antigen peptides of the invention are also encompassed by the invention, as are nucleic acid molecules encoding these antibodies, molecules, fragments or variants.

In a preferred embodiment, one or more of the above combinations are from a single antibody expressing cell line of the invention.

Antibodies of the invention may comprise, or alternatively consist of, a portion (e.g., V_{H} CDR1, V_{H} CDR2, V_{H} CDR3, V_{L} CDR1, V_{L} CDR2, or V_{L} CDR3) of a V_{H} or V_{L} domain having an amino acid sequence of an antibody of the invention or a fragment or variant thereof. In one embodiment, an antibody of the invention comprises, or alternatively consists of, a polypeptide having the amino acid sequence of a V_{H} domain of an antibody of the invention, or a fragment or variant thereof and a V_{L} domain of an antibody of the invention, or a fragment or variant thereof. In another embodiment, an antibody of the invention comprises, or alternatively consists of, a polypeptide having the amino acid sequence of a V_{H} domain and a V_{L} domain from a single antibody (or scFv or Fab fragment) of the invention, or fragments or variants thereof. In one embodiment, an antibody of the invention comprises, or alternatively consists of, a polypeptide having the amino acid sequence of a V_{H} domain of an antibody of the invention, or a fragment or variant thereof. In another embodiment, an antibody of the invention comprises, or alternatively consists of, a polypeptide having the amino acid sequence of a V_{L} domain of an antibody of the invention, or a fragment or variant thereof. In a preferred embodiment, an antibody of the invention comprises, or alternatively consists of, a polypeptide having the amino acid sequence of a V_{H} CDR3 of an antibody of the invention, or a fragment or variant thereof. In another preferred embodiment, an antibody of the invention comprises, or alternatively consists of, a polypeptide having the amino acid sequence of a V_{L} CDR3 of an antibody of the invention, or a fragment or variant thereof. Nucleic acid molecules encoding these antibodies are also encompassed by the invention.

In other embodiment of the invention, V_{L} and V_{H} domains are expressed in a single cell line. In another embodiment, the V_{H} domain and the V_{L} domain are expressed by two different cell lines.

Antibodies of the present invention may also be described or specified in terms of their cross-reactivity. Antibodies that do not bind any other analog, ortholog, or homolog of the antigen of the present invention are included.

In specific embodiments, antibodies of the present invention cross-react with vertebrate homologs of the antigen peptide and the corresponding epitopes thereof.

In a specific embodiment, the above-described cross-reactivity is with respect to any single specific antigenic or immunogenic peptide, or combination(s) of 2, 3, 4, 5, or more of the specific antigenic and/or immunogenic peptides disclosed herein.

The present invention also provides for nucleic acid molecules, generally isolated, encoding an antibody of the invention (including molecules comprising, or alternatively consisting of, antibody fragments or variants thereof). In a specific embodiment, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of a heavy chain expressed by a cell line expressing an antibody of the invention and a V_{L} domain having an amino acid sequence of a light chain expressed by a cell line expressing an antibody of the invention. In another embodiment, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of a heavy chain expressed by a cell line expressing an antibody of the invention or a V_{L} domain having an amino acid sequence of a light chain expressed by a cell line expressing an antibody of the invention.

The present invention also provides antibodies that comprise, or alternatively consist of, variants (including derivatives) of the antibody molecules (e.g., the V_{H} domains and/or V_{L} domains) described herein, which antibodies immunospecifically bind antigen peptides of the invention. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} domain, V_{H}CDR1, V_{H}CDR2, V_{H}CDR3, V_{L} domain, V_{L}CDR1, V_{L}CDR2, or V_{L}CDR3.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind antigen peptides of the invention).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve antibody production from a cell line.

Alternatively, non-neutral missense mutations may alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind antigen peptides of the invention) can be determined using techniques described herein or by routinely modifying techniques known in the art.

In a specific embodiment, an antibody of the invention (including a molecule comprising, or alternatively consisting of, an antibody fragment or variant thereof), that immunospecifically binds antigen peptides of the invention, comprises, or alternatively consists of, an amino acid sequence encoded by a nucleotide sequence that hybridizes to a nucleotide sequence that is complementary to that encoding one of the V_{H} or V_{L} domains expressed by one or more cell lines expressing an antibody of the invention. Hybridization may occur under stringent conditions, under highly stringent conditions, under or under stringent hybridization conditions which are known to those of skill in the art (see above, and, for example, Ausubel F M et al., at pages 6.3.1-6.3.6 and 2.10.3). Nucleic acid molecules encoding these antibodies are also encompassed by the invention.

Antibodies of the present invention act as antagonists of vertebrate PAMP-20 or fragments or variant thereof, and prevent receptor activation and downstream signaling. Receptor activation (i.e., signaling) may be determined by techniques described herein below or otherwise known in the art. For example, receptor activation can be determined by measuring changes in intracellular Ca²⁺ or cAMP levels or phosphorylation of substrates of specifically activated protein kinases. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described below and, for example in, U.S. Pat. No. 5,939,598 incorporated by reference herein in its entirety. Furthermore, human antibodies may be humanized, as described below.

Additionally, the term “antibody” as used herein encompasses chimeric antibodies that bind antigen peptides of the invention. Chimeric antibodies that bind antigen peptides of the invention for use in the methods of the invention have the binding characteristics of the antibodies described above.

By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody may be derived from a non-human source and cause the antibody to bind antigen peptides of the invention. The non-human source can be any vertebrate source that can be used to generate antibodies to antigen peptides of the invention. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). As used herein, the phrase “immunologically active” when used in reference to chimeric antibodies means a chimeric antibody that binds antigen peptides of the invention.

Polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that immunospecifically binds antigen peptides of the invention, comprises, or alternatively consists of, a V_{H} domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a V_{H} domain of a heavy chain of an antibody of the invention.

In another embodiment, an antibody (including a molecule comprising, or alternatively consisting of, an antibody fragment or variant thereof), that immunospecifically binds antigen polypeptides of the invention, comprises, or alternatively consists of, a VL domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a V_{L} domain of a light chain of an antibody of the invention.

The % identity of a polypeptide is determined by GAP (Needleman and Wunsch) analysis (GCG program) with a gap creation penalty=5, and a gap extension penalty=0.3.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 35%, more preferably at least 40%, more preferably at least 45%, more preferably at least 50%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The antibodies of the invention include derivatives that are modified, i.e., by the covalent attachment of any type of molecule to the antibody. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Any of numerous methods of cleavage may be applied, including cleavage by cyanogen bromide, trypsin, chymotrypsin, papain, V8 protease, NaBH₄, acetylation, formylation, oxidation, reduction, metabolic synthesis in the presence of tunicamycin, etc.

In addition, antibodies of the invention may be chemically synthesized. For example, a peptide corresponding to a portion of a protein can be synthesized by use of a peptide synthesizer. Furthermore, if desired, non-classical amino acids or chemical amino acid analogs can be introduced as substitutions and/or additions into the sequence of one, any, both, several or all of the polypeptides of the complex.

Non-classical amino acids include, but are not limited to, the D-isomers of the common amino acids, fluoro-amino acids, designer amino acids such as beta-methyl amino acids, C gamma-methyl amino acids, N gamma-methyl amino acids, and amino acid analogs in general.

Examples of non-classical amino acids are as described earlier.

The antibodies of the present invention may be used either alone or in combination with other compositions. As described above, the antibodies may further be recombinantly fused to a heterologous polypeptide at the N- or C-terminus or chemically conjugated (including covalent and non-covalent conjugations) to polypeptides or other compositions.

The invention also pertains to immunoconjugates, or antibody-drug conjugates (ADC), comprising an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). Such conugates are useful in methods of treatment (discussed below).

The antibodies of the invention may be from any animal origin including birds and mammals. Preferably, the antibodies are human, murine (e.g., mouse and rat), donkey, sheep, rabbit, goat, guinea pig, camel, horse, or chicken.

### Production of Antibodies

For preparation of polyclonal antibodies, any technique which provides serum or purified polyclonal antibodies known to one of ordinary skill in the art may be used.

Serum may be obtained, following immunization and boost injections, for example from a mouse, by any means, including, but not limited to, by tail bleeding. Antibody may be affinity purified by any means known one of ordinary skill in the art. A polyclonal antibody may also either be salted out, and taken up in an appropriate buffer for therapeutic or diagnostic use, or dialyzed with an appropriate buffer.

For preparation of monoclonal antibodies, any technique which provides antibodies produced by continuous cell line cultures can be used, including as described in U.S. Pat. Appl. No. 20070122405 and U.S. Pat. Appl. No. 20070098718. Examples include the hybridoma technique (Kohler G & Milstein C.), the trioma technique, the human B-cell hybridoma technique (Kozbor et al.), and the EBV-hybridoma technique to produce human monoclonal antibodies (Cole et al.). Furthermore, a monoclonal antibody may be prepared by the techniques described in, for example, Clackson T et al; Marks J D et al; and U.S. Pat. No. 5,514,548, in which a monoclonal antibody is isolated from phage antibody libraries. Still yet, a monoclonal antibody may be made by recombinant DNA methods (e.g., U.S. Pat. No. 4,816,567).

As an alternative to the use of hybridomas, antibodies of the invention may be prepared using recombinant DNA methods. DNA encoding the monoclonal antibodies is readily isolated and sequenced using conventional procedures (e.g., by using oligonucleotide probes that are capable of binding specifically to genes encoding the heavy and light chains of murine antibodies). Hybridoma cells described herein serve as a preferred source of such DNA. Once isolated, the DNA may be manipulated, for example, by introducing point mutations, insertions, deletions, fusing the nucleotide sequence, or fragments thereof, to other genes, or expressing it in heterologous gene expression systems, such as, for example, in host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells (see below). Nucleic acid molecules encoding these antibodies are also encompassed by the invention.

For example, an antibody can be produced in a cell line such as a CHO cell line, as disclosed in U.S. Pat. Nos. 5,545,403; 5,545,405; and 5,998,144. In some embodiments, the antibody or antigen-binding fragment thereof is produced in CHO cells using the GS gene expression system (Lonza Biologics, Portsmouth, N.H.), which uses glutamine synthetase as a marker. See, also U.S. Pat. Nos. 5,122,464; 5,591,639; 5,658,759; 5,770,359; 5,827,739; 5,879,936; 5,891,693; and 5,981,216.

Review articles on recombinant expression in bacteria of DNA encoding the antibody include Skerra A et al. and Phuckthun A.

Various techniques have been developed for the production of antibody fragments. Traditionally, these fragments were derived via proteolytic digestion of intact antibodies (see, e.g., Morimoto K & Inouye K. and Brennan M et al.). However, these fragments can now be produced directly by recombinant host cells. For example, the antibody fragments can be isolated from the antibody phage libraries discussed herein above. Alternatively, Fab′-SH fragments can be directly recovered from E. coli and chemically coupled to form F(ab′)₂ fragments (Carter P et al.). According to another approach, F(ab′)₂ fragments can be isolated directly from recombinant host cell culture. Other techniques for the production of antibody fragments will be apparent to the skilled practitioner.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Antibodies isolated using phage display as described above, can be produced, as a non-limiting example, as follows: after phage selection, the antibody-coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax R L et al; and Better M et al.

Examples of techniques which can be used to produce single-chain Fvs and antibodies include those described in U.S. Pat. Nos. 4,946,778 and 5,258,498; Huston J S et al; Shu L et al; and Skerra A et al. 1988. For in vivo use of antibodies in humans, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison S L; Oi et al; Gillies S D et al; U.S. Pat. Nos. 5,807,715; 4,816,567; and 4,816,397.

### Human Antibodies and Antibody Humanization

Included within the scope of the invention, and useful in practicing the methods of the invention, are de-immunized antibodies that have sequence variations produced using methods described in, for example, Patent Publication Nos. EP 0 983 303 A1, WO 00/34317, and WO 98/52976.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al.).

The scope of the present invention also extends to humanized anti-PAMP antibodies. By “humanized” is intended forms of anti-PAMP antibodies that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity.

Humanized antibodies within the scope, and suitable for use in the methods, of the present invention may, for example, have binding characteristics similar to those exhibited by non-humanized antibodies, such as, for example the EGX-P-E9 monoclonal antibody described herein below.

Humanization can be essentially performed following the method of Winter and co-workers (Jones P T et al; Riechmann L et al; Verhoeyen M et al.), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan E A; Studnicka G M et al; Roguska M A et al.), and chain shuffling (U.S. Pat. No. 5,565,332).

In some instances, residues within the framework regions of one or more variable regions of the human immunoglobulin are replaced by corresponding non-human residues (see, for example, Queen et al. U.S. Pat. No. 5,585,089; U.S. Pat. Nos. 5,693,761; 5,693,762; and 6,180,370; see also, e.g., Riechmann L et al.).

Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence.

The humanized antibody optionally also will comprise at least a portion of an immunoglobulin constant region (Fc), typically that of a human immunoglobulin. For further details see Jones P T et al; Riechmann L et al; and Presta. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160 where humanized antibodies and techniques for producing humanized antibodies having improved affinity for a predetermined antigen are disclosed.

### XenoMouse™ Technology

Human antibodies can also be produced using transgenic animals which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes.

For an overview of this technology for producing human antibodies, see Lonberg N and Huszar D. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598.

In one embodiment, antibodies of the invention are prepared by the utilization of a transgenic animal(s) (e.g., XenoMouse™ strains available from Abgenix Inc., Fremont, Calif.). Accordingly, the antibodies of the invention extend to xenogeneic or modified antibodies produced in a non-human mammalian host.

In another embodiment, fully human antibodies are obtained by immunizing transgenic mice. One such mouse is obtained using XenoMouse™ technology (Abgenix; Fremont, Calif.), and is disclosed in U.S. Pat. Nos. 6,075,181, 6,091,001, and 6,114,598. Fully human antibodies are expected to minimize the immunogenic and allergic responses intrinsic to mouse or mouse-derivatized monoclonal antibodies and thus to increase the efficacy and safety of the administered antibodies.

In another embodiment, the antibody of the invention is a humanized version of the EGX-P-E9 monoclonal antibody.

### Antibody Optimisation

Included with the scope of the invention is an anti-PAMP antibody which has been affinity matured.

An “affinity matured” antibody is one with one or more alterations in one or more CDRs thereof which result in an improvement in the affinity of the antibody for the PAMP antigen, compared to a parent antibody which does not possess those alteration(s). Preferred affinity matured antibodies will have nanomolar or even picomolar affinities for the target antigen. Affinity matured antibodies are produced by procedures known in the art. Marks et al. describes affinity maturation by V_{H} and V_{L} domain shuffling. Random mutagenesis of CDR and/or framework residues is described by, for example Barbas et al. 1994; and Jackson et al. 1995.

Also included within the scope of the invention are “veneered antibodies”. The term “venerred antibody” refers to the selective replacement of framework region residues from, for example, a mouse heavy or light chain variable region with human framework region residues in order to provide a xenogeneic molecule comprising comprising an antigen-binding site which retains substantially all of the native framework region folding structure. Veneering techniques are based on the understanding that the ligand-binding characteristics of an antigen-binding site are determined primarily by the structure and relative disposistion of the heavy and light chain CDR sets within the antigen-binding surface (Davies et al.). Thus, antigen-binding specificity can be preserved in a humanized aqntibody only wherein the CDR structures, their interaction with eachother, and their interaction with the rest of the V region domains are carefully maintained. By using venerring techniques, exterior (e.g. solvent accessible) framework region residues, which are readily encountered by the immune system, are selectively replaced with human residues to provide a hybrid molecule that comprises either a weakly immunogenic, or substantially non-immunogeneic, venerred surface.

The process of veneering makes use of the available sequence date for human antibody variable domains compiled by Kabat et al. Solvent accessibilities of V region amino acids can be deduced from the known three-dimensional structure for human and non-human antibody fragments.

There are two general steps in veneering a non-human antigen-binding site. Initially, the framework regions of the variable domains of an antibody molecule of interest are compared with corresponding framework region sequences of human variable domains available databases. The most homologous human variable regions are then compared residue by residue to corresponding non-human amino acids. The residues in the non-human framework region that differ from the human counterpart are pleced by the residues present in the human moiety using recombinat techiques known in the art. Residue switching is carried out with moieties that are at least partially exposed (solvent accessible), and care is exercised in the replacement of amino acid residues that may have a significant effect on the tertiary structure of variable region domains, such as proline, glycine and charged amino acids.

In this manner, the resultant “veneered” non-human antigen-binding sites are thus designed to retain the non-human CDR residues, the residues substantially adjacent to the CDRs, the residues identified as buried or mostly buried (solvent inaccessible), the residues believed to participate in non-covalent (e.g. electrostatic and hydrophobic) contact between heavy and light chain domains, and the residues from conserved structural regions of the framework regions which are believed to influence the “canonical” tertiary structures of the CDR loops. These design criteria are then used to prepare recombinant nucleotide sequences that combine the CDRs of both the heavy and light chain of a non-human antigen-binding site into human-appearing framework regiosn that can be used to transfect mammalian cells for the expression of recombinant human antibodies that exhibit the antigen-binding specificity of the non-human antibody molecule.

Included within the scope of the invention, are antibodies wherein the Fc portion has been modified to enhance the effector function(s) of the antibody such as complement mediated cytotoxicity (CDC) or antibody dependent cellular cytotoxicity (ADCC).

Modified glycoforms of antibodies of the present invention may be useful for a variety of purposes, including but not limited to enhancing or reducing effector function and/or modifying half life of the antibody (see, for example, WO/2007/010401). Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Alterations may results in a decrease or increase of CIq binding and CDC or of FcγR binding and ADCC. Substitutions can, for example, be made in one or more of the amino acid residues of the heavy chain constant region, thereby causing an alteration in an effector function while retaining the ability to bind to the antigen as compared with the modified antibody, cf. U.S. Pat. No. 5,624,821 and U.S. Pat. No. 5,648,260. Engineered glycoforms may be generated by any method known to one skilled in the art, for example by using engineered or variant expression strains, by co-expression with one or more enzymes, for example β(1,4)—N-acetylglucosaminyltransferase III (GnTIl 1), by expressing an antibody or fragment thereof in various organisms or cell lines from various organisms, or by modifying carbohydrate(s) after the antibody or fragment has been expressed. Methods for generating engineered glycoforms are known in the art and include but are not limited to those described in U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

ADCC and/or CDC activity may be enhanced by introducing one or more amino acid substitutions in an Fe region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al. Homodimeric antibodies with enhanced anti-tumor activity may also be prepared using heterobifunctional cross-linkers. Alternatively, an antibody can be engineered which has dual Fe regions and may thereby have enhanced complement lysis and ADCC capabilities.

### Assays

### (i) Antibody Binding

The antibodies of the invention may be assayed for immunospecific binding by any method known in the art. The immunoassays which can be used include but are not limited to competitive and non-competitive assay systems using techniques such as BIAcore analysis, FACS (Fluorescence activated cell sorter) analysis, immunofluorescence, immunocytochemistry, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and known in the art (see, e.g., Ausubel et al,). Exemplary immunoassays are described briefly below, but are not intended by way of limitation.

Antibodies that competitively inhibit the anti-PAMP antibodies of the invention may be identified by their ability to compete with EGX-P-E9 for binding to PAMP. In this procedure the PAMP sequence may be conjugated with biotin using established procedures (Hoffman K et al.). Competitive antibodies are then evaluated by their capacity to compete with the binding of biotinylated EGX-P-E9 to PAMP. The binding may be assessed by the addition of fluorescein-labelled streptavidin which will bind to biotin on the PAMP sequence. Fluorescence staining of cells is then quantified by flow cytometery, and the competitive effect of the competitor antibody expressed as a percentage of the fluorescence levels obtained in the absence of competitor.

In a preferred embodiment, the antibody competitively inhibits binding to the PAMP epitope by at least 95%, at least 90%, at least 85%, at least 80%, at least 75%, at least 70%, at least 60%, or at least 50%.

Examples of suitable immunoassays include immunoprecitation protocols, Western Blot analysis and ELISAs. Further discussion of these protocols can be found in for example, Ausubel et al, at 10.16.1, 10.8.1 and 11.2.1 respectively.

The binding affinity of an antibody to an antigen and the off-rate of an antibody-antigen interaction can be determined by any method known to one of ordinary skill in the art, such as competitive binding assays. One example of such an assay is a radioimmunoassay.

In a preferred embodiment, BIAcore kinetic analysis is used to determine the binding on and off rates of antibodies (including antibody fragments or variants thereof) to antigen of the current invention. BIAcore kinetic analysis comprises analyzing the binding and dissociation of antibodies from chips with immobilized antigen on their surface.

### (ii) Assays Specific for Antibodies of the Instant Invention

Antibodies of the present invention may be used, for example, to purify, detect, and target the peptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al.).

The present invention provides for antibodies (including molecules comprising, or alternatively consisting of, antibody fragments or variants thereof), that neutralize PAMP activity. An antibody that “neutralizes the activity of PAMP or a fragment or variant thereof” is, for example, an antibody that diminishes or abolishes the ability of PAMP or a fragment or variant thereof to bind to and/or activate its receptor; that abolishes or inhibits PAMP signaling (e.g., calcium flux initiated by an activated G-protein coupled receptor and/or cAMP production); that diminishes or abolishes PAMP-induced physiological effects, such as, for example, modulation of gene expression.

In one embodiment of the present invention, antibodies (including molecules comprising, or alternatively consisting of, antibody fragments or variants thereof) reduce or abolish the ability of PAMP to modulate intracellular signaling pathways, such as, for example, but not limited to, affecting Ca2⁺ influx and/or cAMP production, as determined by any method known in the art.

In another embodiment, the present invention also provides for antibodies (including molecules comprising, or alternatively consisting of, antibody fragments or variants thereof), that reduce, inhibit, or prevent up- or down-regulation of the transcription, translation, or secretion of PAMP, AM, steroids, c-fos, or any other genes or gene products of which expression is regulated by PAMP-induced signalling.

In another embodiment, the present invention also provides for antibodies (including molecules comprising, or alternatively consisting of, antibody fragments or variants thereof), that reduce, inhibit, or prevent induction of any PAMP-induced hypotensive effect(s), or induction of any PAMP-induced vasorelaxant effect(s).

In yet another embodiment of the present invention, antibodies (including molecules comprising, or alternatively consisting of, antibody fragments or variants thereof) reduce or abolish the ability of PAMP to modulate cell growth, such as, for example, but not limited to, induction of vascular endothelial cell replication, or reduce or abolish the ability of PAMP to induce cellular migration, such as vascular cell migration (e.g. as a part of angiogenesis).

In yet another embodiment of the present invention, antibodies (including molecules comprising, or alternatively consisting of, antibody fragments or variants thereof) reduce or abolish the ability of PAMP to induce angiogenesis.

Preferably, the antibody according to the invention, upon binding to PAMP is capable of inducing one or more of the following biological activities:

1. Inibition of PAMP receptor activation
2. Inhibition of PAMP-induced changes in cellular Ca²⁺ influx
3. Inhibition of PAMP-induced changes in cAMP production
4. Inhibition of PAMP-induced up- or down regulation of gene expression (e.g. fos, catecholamine synthesizing enzymes)
5. Reversal of PAMP-induced inhibition of hormone secretion
6. Inhibition of PAMP-induced hypotensive effect
7. Inhibition of PAMP-induced vasorelaxant effect
8. Reversal of PAMP-induced modulation of cell growth
9. Inhibition of PAMP-induced cellular migration
10. Inhibition of PAMP-induced angiogenic effect
11. Inhibition of PAMP-induced proliferation of cancer cells in vivo.

Any method know to one of ordinary skill in the art can be used to assay inhibition of binding of PAMP, or any fragments, variants, or derivatives to receptors in the presence and absence of antibodies of the present invention.

Assaying receptor activation may be achieved by any method known to one of ordinary skill in the art, such as by monitoring cellular responses to PAMP, or the absence of such responses in the presence of an antibody or antigen-binding fragment thereof which prevents the activation by PAMP of PAMP receptors on cells.

Inhibition of Ca²⁺ Influx and cAMP Production

PAMP reduces carbachol-induced Ca²⁺ influx in endothelial cells (Martínez 2004), so an antibody that prevents PAMP receptor activiation should restore carbachol-induced Ca²⁺ influx. Using the assay described by Martínez 2004 would allow for the identification of an antibody of fragment thereof that prevents PAMP from activating its receptor.

Using Ca²⁺ ionophores, such as, for example, A23187 (see for example Flaherty M et al. 1986, and the Ca2+-channel agonists, such as, for example, but not limited to, BAYK-8644 (Belloni A S et al. 1999), carbachol (Katoh F et al.), and ATP, Ca²⁺ influx can be monitored in the presence and absence of PAMP and in the presence and absence of antibody by any means known to one of ordinary skill in the art, such as, for example by using a flurescent plate reader system as described in Nothacker H-P et al. For example, human dermal microvascular endothelial cells may be cultured in 96-well plates at 1.0×10⁵ cells per well. The cells may be loaded for an appropriate period of time, such as, for example, 60 minutes, at an appropriate temperature, such as, for example, room temperature, with a fluorescent dye, such as, for example, but not limited to, FLIPR (Molecular Devices, Sunnyvale, Calif.) and then transferred to a FlexStation II (Molecular Devices) for analysis. Test compounds may be prepared in another plate at 5× concentration and added to the proper wells by the robotic arm of the FlexStation II. Fluorescence can be measured every 5 seconds in each well and recorded.

Inhibition of PAMP-induced cAMP production and/or release by an antibody of the present invention in cells responsive to PAMP, or any fragment, variant, fusion, or derivative thereof, may be assayed by any method known to one of ordinary skill in the art, as, for example described in Isobe et al., 1993 (Isobe K. et al.), in the presence and absence of PAMP, or a fragment, variant, fusion, or derivative thereof, in the presence and absence of antibody. Non-limiting examples of cells responsive to PAMP-20 are listed above.

For example, released cAMP levels may be assayed in the medium, and intracellular cAMP levels may be assayed, before and after exposure of cells responsive to PAMP, or any fragment, variant, fusion, or derivative thereof, may be assayed by any method known to one of ordinary skill in the art, in the presence and absence of PAMP, or a fragment, variant, fusion, or derivative thereof, in the presence and absence of antibody. Cells may, for example, be incubated at 37 degrees C. for 20 minutes with the reagents described above, and at the end of the 20-min incubation, cells may be harvested and/or the medium may be collected, and each or both stored at −20 degrees C. or −70 degrees C. until assayed. Cyclic AMP may measured by competitive protein binding assay. Cellular protein content may be assayed by standard methods known to one of ordinary skill in the art. Furthermore, commercially available cAMP radio immuno assays kits, such as for example the kit available from NEN Life Sci. Products Inc. (Boston, Mass.) may be used according to standard procedures known to one of ordinary skill in the art.

Alternatively, cells may be washed (twice) with medium (e.g. Eagle's minimal essential medium) and preincubated in medium containing 0.2 mM 3-isobutyl-1-methylxanthine (IBMX) for 5 min. Experiments may be initiated by replacing the medium weth HEPES-buffered Krebsbuffer including test substances and 0.2 mM IBMX, and the cells are then incubated at 37 degrees C. for 30 min. The reaction is terminated by adding 100 micro-L of 1 N HCl followed by incubation on ice for 30 min. The cAMP in the acid extract is then measured with a commercially available cAMP kit (e.g. Yamasa, Chousi, Japan).

### Modulation of Gene Expression

Inhibition of PAMP-induced alterations modulation in gene expression by an antibody of the present invention in cells responsive to PAMP, or any fragment, variant, fusion, or derivative thereof, may be assayed by any method known to one of ordinary skill in the art, in the presence and absence of PAMP, or a fragment, variant, fusion, or derivative thereof, in the presence and absence of antibody. Non-limiiting examples of cells responsive to PAMP are listed above.

Analysis of gene expression may focus, as non-limiting examples, on transcription, translation, or sectretion of the pre-proadrenomedullin gene in cells expressing AM or PAMP or both (see Qi Y F et al.), the catecholamine-synthesizing enzymes tyrosine hydroxylase and dopamine beta-hydroxylase in PC12 cells (see Takekoshi K et al.), and c-fos in neuronal cells and tissues (Ueta Y et al.), by any means known to one of ordinary skill in the art, such as, for example, but not limited to, northernblot analysis, immunohistochemistry, westernblot analysis, radioimmunoassay, and quantitative RT-PCR.

For example, primary culture of vascular smooth muscle cells (VSMC) from (rat) aorta may be incubated in various amounts of PAMP, preferably 10⁻⁷M, for various periods of time, for example, but not limited to, time course experiment ranging from 1 min to 72 hours, and pre-preadrenomedullin gene expression may be analyzed, as described in detail in Qi et al. 2002 (Qi Y F et al.). procedures known to one of ordinary skill in the art. Six microliters PCR product may be separated in a 1.5% agarose gel, and stained, for example, with ethidium bromide. Ratio of optical density of the analyte and internal standard bands may be measured, for example, but not limited to, using the Gel Documentation System (Bio-Rad, Hercules, Calif.). Amplification of analyte cDNA and the internal competitive standard maybe confirmed by digestion of the PCR products with appropriate restriction enzymes (e.g. single cutters). To calibrate the sample loaded in quantitative PCR, beta-actin cDNA may be determined at the same time. Two microliters of PCR product may be amplified again using any beta-actin-specific forward and reverse primers for 20 cycles at temperatures and for periods assessed based on the nucleotide sequences of the primers according to standard procedures known to one of ordinary skill in the art, and the optical density of the beta-actin band may then be measured. Relative amount of beta-actin cDNA in loaded sample may be obtained from a beta-actin standard curve. Calibrated amount of analyte mRNA may also be used for further analysis.

As additional examples of assays suitable for rate of transcription analysis, real-time PCR and Northern Blot analysis, using, for example, (³²P-labelled) probes (labeled, for Northern Bot analysis, as a non-limiting example, according to standard ‘random primer extension’ procedures know to one of ordinary skill in the art) may also be applied for transcript quantification according to standard procedures known to one of ordinary skill in the art as described in detail in Martínez et al., 2004.

Translation rates and cellular protein synthesis/production may be assayed by standard methods known to one of ordinary skill in the art. As non-limiting examples, protein levels may be determined by Western Blot analysis and by immunohistochemistry, for example, where the location of the protein within a given tissue is of significance (see above).

### Inhibition of Hormone Release

Inhibition of PAMP-induced alterations in hormone synthesis and release in cells responsive to PAMP by an antibody of the present invention, or any fragment, variant, fusion, or derivative thereof, may be assayed by any method known to one of ordinary skill in the art, in the presence and absence of PAMP, or a fragment, variant, fusion, or derivative thereof, in the presence and absence of antibody.

Examples of assays that can be applied in the context of the present invention include those described in detail by Belloni A S et al; Shimosawa T et al 1997; and Shimosawa T et al. 1995.

For example, inhibition of aldosterone secretion by human adrenocortical and Conn's adenoma cells by an antibody of the present invention may be assayed in the presence and absence of PAMP, or a fragment, variant, fusion, or derivative thereof, in the presence and absence of antibody as described in detail in Andreis et al., 1998 (Andreis P G et al.).

Inhibition of PAMP's anti-cholinergic effect, i.e. inhibition of catecholamine secretion in adrenal medulla upon choninergic stimulation (see above), by an antibody of the present invention can be assayed according to the procedures described in detail in Katoh et al., 1995, whereby antibody is added in order to assay its PAMP-inhibitory effect.

As another example, inhibition of PAMP's stimulatory effect on DHEA secretion in H295R cells by an antibody of the present invention may be assayed in the presence and absence of PAMP, or a fragment, variant, fusion, or derivative thereof, in the presence and absence of antibody as described in detail in Thomson et al., 2003 (Thomson L M. et al.).

To determine the effect of PAMP, or a fragment, variant, fusion, or derivative thereof, in the presence and absence of an antibody of the present invention on steroid secretion, cells cultured in six-well plates which have not been pretreated are maintained overnight (˜16 h) in +1 ITS-free (i.e., without medium supplement), Ultroserfree medium, commercially available, for example, from Universal Biologicals (Gloucester, UK), then washed twice with phosphate-buffered saline before being incubated for various periods of time, such as, for example, but not limited to in time-course experiments or 4 h, preferably at 37 degrees C., with either no agonist (control), adrenomedullin (10 μmol/l to 100 nmol/l), PAMP or a fragment, variant, fusion, or derivative thereof, in the presence and absence of an antibody of the present invention.

At the end of the incubation period, the medium is removed to fresh tubes and stored at −20 degrees C. Aldosterone and cortisol may be assayed by any method known to one of ordinary skill in the art. DHEA concentrations may be assayed following the manufacturer's instructions of a commercially available kit (available, for example, from EuroDPC, Llanberis, UK). Cells may be scraped off for protein content determination by any method known to one of ordinary skill in the art, including the method of Lowry.

As another example, inhibition of PAMP's stimulatory effect on histamine release from rat peritoneal mast cells by an antibody of the present invention may be assayed in the presence and absence of PAMP, or a fragment, variant, fusion, or derivative thereof, in the presence and absence of antibody as described in detail in Yoshida et al., 2001.

### Inhibition of PAMP's Hypotensive Effect

Inhibition of PAMP-induced hypotensive effect, such as cardiovascular and sympathetic effects, by an antibody of the present invention, or any fragment, variant, fusion, or derivative thereof, may be assayed by any method known to one of ordinary skill in the art, in the presence and absence of PAMP, or a fragment, variant, fusion, or derivative thereof, in the presence and absence of antibody.

For example, such assays may focus on the effects of intravenously administered PAMP, or any fragment, variant, fusion, or derivative thereof, on mean arterial pressure, heart rate, and renal sympathetic nerve activity, and on the arterial baroreceptor reflex.

In another example, such assays may focus on the inhibitory effects of intravenously administered antibodies on effects of co-administered PAMP, or any fragment, variant, fusion, or derivative thereof, in the presence and absence of a Gi protein inhibitor, pertussis toxin, on norepinephrine overflow from sympathetic nerve endings, plasma norepinephrine levels and blood pressure change, e.g. in pithed rats, and response of blood pressure and heart rate in unanesthetized and unrestrained animals, as described in detail in Shimosawa et al., 1997 (Shimosawa T et al. 1997).

To assay responses of blood pressure and heart rate to PAMP, or a fragment, variant, fusion, or derivative thereof, in the presence and absence of antibody are administered to unanesthetized and unrestrained experimental animals, such as male adult Wistar rats, animals from each group are used, as described above, 1 day after the last injection of PTX or saline. The animals are anesthetized with ether, preferably twenty four hours before the experiments are carried out, and the surgical procedures and cannula implants, except for tracheal cannula, are done as described above. Both catheters are plugged with stainless steel pins. After the surgical procedure, the rats are placed in (a) cage(s) that permit(s) free movement for at least 24 hours to acclimate them to the new environment. Mean arterial pressures and heart rates are recorded with a pressure transducer (e.g. model TP-200T, Nihon Kohden) connected to a thermal array recorder (e.g. model WS-641G Nihon Kohden). PAMP (e.g. 40 nmol/kg) or a fragment, variant, fusion, or derivative thereof, in the presence and absence of antibody, and preferably also in the presence and absence of a PAMP antagonist, such as PAMP (12-20), and inhibitors ofintracellular signaling molecules (e.g. G proteins and PKA) is dissolved, for example, in 0.1 mL of saline and preferably injected as an intravenous bolus into the jugular vein in random order. Another injection may be done 15 minutes after mean arterial pressue and heart rate return to basal level.

To examine the efficacy of pertussis toxin administration, carbachol (e.g. 1, 3, 10, 30, and 100 mg per rat) preferably dissolved in 0.1 mL of saline, is injected as a bolus 1 hour after PAMP, or a fragment, variant, fusion, or derivative thereof, in the presence and absence of antibody is administered, and the decreases in heart rate are recorded. Changes in mean arterial pressure and heart rate are measured at nadir level.

### Inhibition of PAMP's Vasorelaxant Effect

Inhibition of PAMP-induced vasorelaxant effects, such as vasodilator and sensory effects, by an antibody of the present invention, or any fragment, variant, fusion, or derivative thereof may be assayed, as a non-limiting example, in human skin, by any method known to one of ordinary skill in the art, in the presence and absence of PAMP, or a fragment, variant, fusion, or derivative thereof, in the presence and absence of said antibody e.g. by intradermal injection of the forearm.

Such methods include those described in detail in Hasbak et al., 2006; and Nakamura et al.

### Modulation of Cellular Growth

Inhibition of PAMP-induced modulation of cellular division rates by an antibody of the present invention, or any fragment, variant, fusion, or derivative thereof may be assayed by any method known to one of ordinary skill in the art, in the presence and absence of PAMP, or a fragment, variant, fusion, or derivative thereof, in the presence and absence of said antibody of the present invention.

### Microvascular Endothelial Cell Proliferation

Inhibition of stimulation of microvascular endothelial cell proliferation is assayed according to any methods known to one of ordinary skill in the art. As a non-limiting example of such methods, the assays described in detail in Martínez et al., 2002 (Martínez A et al. 2002) may be applied. Accordingly, microvascular endothelial cell are seeded, for example, in 96-well plates at a density of 2.0×10⁵ cells per well in serum-free medium containing different concentrations of the test peptides/agents. After 3 days in culture, the number of viable cells per well is estimated by the 3-(4,5-dimethylthiazol-2-yl)-2,5-diphenyltetrazolium bromide assay as described in detail in Martínez A et al. 2002. Results are preferably represented as the percentage of growth over the untreated control.

### Inhibition of PAMP-Induced Cell Migration

Inhibition of PAMP-induced migration of endothelial cells is assayed according to any methods known to one of ordinary skill in the art. As a non-limiting example of such methods, the assay described in detail in Martínez et al., 2004 (Martínez A et al. 2004. Cancer Research 64: 6489-6494, incorporated by reference herein) may be applied. Accordingly, cell motility is measured as described in detail in Martínez et al., 2002. PAMP, or a fragment, variant, fusion, or derivative thereof, in the presence and absence of an antibody of the present invention is placed at various concentrations at the bottom of a chemotaxis chamber (e.g. NeuroProbe, Inc., Gaithersburg, Md.). The intermediate membrane is coated with 10 micro-g/mL fibronectin, and in the upper chamber, for example, 5.0×10⁵, human endothelial cells are added. After an incubation, preferably for 4-hour at 37 degrees C., the membrane is fixed and stained (e.g. Protocol Hema3; Biochemical Sciences, Inc., Bridgeport, N.J.). The cells trapped in the porous membrane are photographed through an x25 microscope objective, and the number of cells per photographic field is counted.

### Inhibition of Angiogenesis

Inhibition of PAMP-induced angiogenesis by an antibody of the present invention is assayed according to any methods known to one of ordinary skill in the art. As a non-limiting example of such methods, the assays described in detail in Martínez et al., 2004; Auerbach et al., 2003, Martínez et al., 2002.

For example, the chick embryo aortic arch assay may be carried out, which is an ex vivo angiogenesis assay that is described in detail in Isaacs et al., 2002, Auerbach et al., 2003. In brief, aortic rings of approximately 0.8 mm in length are prepared from the five aortic arches of 13-day-old chicken embryos (available, for example, from CBT Farms, Chestertown, Md.), and the soft connective tissue of the adventitia layer is carefully removed with tweezers. Each aortic ring is placed in the center of a well, for example, in a 48-well plate and preferably covered with 10 micro-L of Matrigel (BD Biosciences, San Jose, Calif.). After the Matrigel solidifies, 300 micro-L of growth-factor free medium, preferably growth factor-free human endothelial-SFM basal growth medium (Invitrogen, Carlsbad, Calif.), containing the proper concentration of the test substances (PAMP, or a fragment, variant, fusion, or derivative thereof, in the presence and absence of an antibody of the present invention are added to each well. The plates are preferably kept in a humid incubator at 37 degrees C. in 5% CO₂ for 24 to 36 hours.

As another example, analysis and quantitation of angiogenesis may be carried out using the directed in vivo angiogenesis assay as described in detail in Martínez et al., 2002; and Guédez et al., 2003. In brief, surgical-grade silicone tubes, preferably 10-mm-long, with only one end open (angioreactors) are filled preferably with 20 micro-L of Matrigel alone or mixed with bFGF or VEGF as positive controls, or with PAMP, or a fragment, variant, fusion, or derivative thereof, in the presence and absence of an antibody of the present invention at the various concentrations. After the Matrigel solidifies, the angioreactors are implanted into the dorsal flanks of athymic nude mice (e.g. those from the NCl colony). After approximately 11 days, the mice receive i.v. injections of 25 mg/mL FITC-dextran (100 micro-L/mouse; available, for example, from Sigma, St. Louis, Mo.) 20 minutes before removing angioreactors. Photographs of the implants are taken for visual examination of angiogenic response. Quantitation of neovascularization in the angioreactors is determined as the amount of fluorescence trapped in the implants and is measured, for example, in an HP Spectrophotometer (Perkin-Elmer, Boston, Mass.).

As yet another example, cord formation assays may be carried out. Accordingly, human endothelial cells are seeded, preferably at 2.0×10⁵ cells per well, over a solid layer of Matrigel covering the bottom of, for example, a 24-well plate in the presence or absence of PAMP, or a fragment, variant, fusion, or derivative thereof, in the presence and absence of an antibody of the present invention. After an overnight incubation, the tubular structures are photographed, and the number of knots per photographic field are counted as a measure of lattice complexity.

### Inhibition of PAMP-Induced Proliferation of Cancer Cells In Vivo

Because angiogenesis—the growth of new blood vessels—is required for optimal growth of solid tumors beyond very small masses, and since PAMP is an angiogenesis-promoting factor in many solid tumors (Martínez, 2004), an antibody, or fragment thereof, of the present invention may be assayed for its ability to reduce tumor growth rates as an assay for preventing its ability to activate its receptors.

For example, xenograft experiments may be carried out, as described in Martínez et al, 2004. Accordingly, female athymic nude mice, for example, from the NIH colony in Frederick, Md., may be given s.c. injections of 1.0×10⁷ A549 cells per mouse. Approximately two weeks later, all of the mice will have developed palpable tumors under the skin, and at this time, they are randomly divided in several groups. Three times a week, each individual tumor is measured (length, height, and thickness), and every mouse will receive an intratumoral injection of antibodies, or fragments thereof, to determine whether they interfere with PAMP-promoted tumor growth. This approach is based on reported antiangiogenic treatments in xenograft studies (Martínez, 2004; Ouafik L et al.).

### Therapeutic Uses

The present invention is further directed to antibody-based therapies which involve administering antibodies of the invention to an animal, preferably a mammal, and most preferably a human for treating one or more disorder or conditions mediated by PAMP.

Therapeutic compounds of the invention include antibodies of the invention (including fragments, variants, analogs, fusions, and derivatives thereof as described herein) and nucleic acids encoding antibodies of the invention (including fragments, analogs and derivatives thereof).

The antibodies of the invention can be used to treat, inhibit or prevent diseases, disorders or conditions associated with aberrant expression and/or activity of PAMP; alternatively, the antibodies of the invention can be used to treat, inhibit or prevent diseases, disorders or conditions associated with aberrant physiology that can be corrected by therapeutic application of the antibodies of the present invention, including, but not limited to, any one or more of the diseases, disorders, or conditions described herein.

As used herein, the terms “treating”, “treat” or “treatment” include administering a therapeutically effective amount of an anti-PAMP antibody or antigen-binding fragment thereof described herein sufficient to reduce or eliminate at least one symptom of a specified disease or condition.

As used herein, the terms “preventing”, “prevent” or “prevention” include administering a therapeutically effective amount of anti-PAMP antibody or antigen-binding fragment thereof described herein sufficient to stop or hinder the development of at least one symptom of a specified disease or condition.

The treatment and/or prevention of diseases, disorders, or conditions associated with aberrant expression and/or activity of PAMP includes, but is not limited to, alleviating symptoms associated with those diseases, disorders or conditions. Antibodies of the invention may be provided in pharmaceutically acceptable compositions as known in the art or as described herein.

The antibodies of the present invention may be used therapeutically to bind PAMP peptides according to the invention locally or systemically in the body. With the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or fragments, variants, derivatives of antibodies of the invention, or with lymphokines or hematopoietic growth factors, or small molecule therapeutics useful in the treatmenst of the diseases, disorders or conditions that may be addressed.

The antibodies of the invention may be administered alone or in combination with other types of treatments (e.g., radiation therapy, chemotherapy, hormonal therapy, immunotherapy, or anti-tumor agents). Generally, administration of products of a species origin or species reactivity (in the case of antibodies) that is the same species as that of the patient is preferred. Thus, in a preferred embodiment, human or humanized antibodies, fragments, variants, derivatives, or analogs are administered to a human patient for therapy or prophylaxis.

It is preferred to use high affinity and/or potent in vivo inhibiting and/or neutralizing antibodies against PAMP. The preferred binding affinities are as described earlier.

As used herein, “a therapeutically effective amount” means an amount required to achieve a desired end result weighted against any toxic or detrimental effects. The amount required to achieve the desired end result will depend on the nature of the specific antibody, which can be determined as described above without undue experimentation, and the diseases, conditions, or disorders being treated, and can be determined by standard clinical techniques. In addition, in vitro assays may optionally be employed to help identify optimal dosage ranges. The precise dose to be employed will also depend on the route of administration and the seriousness of the disease or disorder, and should be decided according to the judgment of the practitioner and each subject's circumstances. Effective doses may be extrapolated from dose-response curves derived from in vitro or animal model test systems.

Various delivery systems are known and can be used to administer a pharmaceutical composition of the present invention. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, but not limited to, local infusion during surgery, by injection, by means of a catheter, or by means of an implant, said implant being of a porous, non-porous, or gelatinous material, including membranes, such as sialastic membranes, or fibers.

In one embodiment, administration can be by direct injection at the site (or former site) of a malignant tumor or neoplastic or pre-neoplastic tissue.

In one embodiment, a pump may be used (see Langer and Saudek et al.).

In another embodiment, a controlled release system can be placed in proximity of the therapeutic target, i.e., the brain, thus requiring only a fraction of the systemic dose (see, e.g., Goodson, 1984).

Other controlled release systems are discussed in the review by Langer.

In a preferred embodiment, the antibody of the present invention is formulated in accordance with routine procedures as a pharmaceutical composition adapted for intravenous administration to human beings. Typically, compositions for intravenous administration are solutions in sterile isotonic aqueous buffer. Where necessary, the antibody of the present invention may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the antibody of the present invention is to be administered by infusion, it can be dispensed with an infusion bottle containing sterile pharmaceutical grade water or saline. Where the antibody of the present invention is administered by injection, an ampoule of sterile water for injection or saline can be provided so that the ingredients may be mixed prior to administration.

### Considerations for Pharmaceutical Compositions

Antibodies of the invention are preferably administered in a pharmaceutically acceptable carrier.

The term “pharmaceutically acceptable” means approved by a regulatory agency of the Federal or a state government or listed in the U.S. Pharmacopeia or other generally recognized pharmacopeia or receiving specific or individual approval from one or more generally recognized regulatory agencies for use in animals, and more particularly in humans.

The term “carrier” refers to a diluent, adjuvant, excipient, or vehicle with which the therapeutic is administered. Such pharmaceutical carriers can be sterile liquids, such as water, organic solvents, such as certain alcohols, and oils, including those of petroleum, animal, vegetable or synthetic origin, such as peanut oil, soybean oil, mineral oil, sesame oil and the like. Buffered saline is a preferred carrier when the pharmaceutical composition is administered intravenously. Saline solutions and aqueous dextrose and glycerol solutions can also be employed as liquid carriers, particularly for injectable solutions.

The composition, if desired, can also contain minor amounts of wetting or emulsifying agents, or pH buffering agents. These compositions can take the form of solutions, suspensions, emulsion and the like. Examples of suitable pharmaceutical carriers are described in “Remington's Pharmaceutical Sciences” by E. W. Martin. Such compositions will contain a therapeutically effective amount of the therapeutic antibody of the invention, preferably in purified form, together with a suitable amount of carrier so as to provide the form for proper administration to the patient. The formulation should suit the mode of administration.

In a preferred embodiment, the antibody of the present invention is formulated in accordance with routine procedures as a pharmaceutical composition adapted for intravenous administration to human beings. Typically, compositions for intravenous administration are solutions in sterile isotonic aqueous buffer.

### Kits

The present invention also provides therapeutic/diagnostic kits comprising anti-PAMP antibodies, and/or antigen-binding fragments thereof of the present invention for use in the present treatment methods. Such kits will generally contain, in suitable container means, a pharmaceutically acceptable formulation of at least one anti-PAMP antibody, and/or antigen-binding fragments thereof. The kits may also contain other pharmaceutically acceptable formulations, either for diagnosis/imaging or combined therapy. For example, such kits may contain any one or more of a range of anti-angiogenic agents; and/or anti-tumor cell antibodies

The containers of the kit will generally include at least one vial, test tube, flask, bottle, syringe or other container means, into which the anti-PAMP antibodies, and/or antigen-binding fragments thereof and any other desired agent, may be placed and, preferably, suitably aliquoted. The kits may also comprise a further container means for containing a sterile, pharmaceutically acceptable buffer or other diluent.

The kits may also contain a means by which to administer the anti-PAMP antibodies, and/or and/or antigen-binding fragments thereof of the present invention to a subject.

Optionally, the kit may also comprise instructions for use.

The invention is hereinafter described by way of the following non-limiting Examples and with reference to the accompanying figures.

### Example 1

### Isolation and Characterisation of the EGX-P-E9 Antibody

### (i) Immunization

Full length PAMP, C-terminally amidated and containing an additional N-terminal cysteine residue was synthesized and conjugated to both KLH and bovine serum albumin (BSA). The KLH conjugate was used to immuize Balb/c mice three times subcutaneously over a two month period. The first immunization was performed with 50 μg of PAMP-KLH per mouse, mixed with Complete Freund Adjuvant and further immunizations were performed with Incomplete Freund Adjuvant (IFA). Sera titer was determined by ELISA using immobilized PAMP-BSA, detected using an anti-mouse antibody-HRP conjugate, after the last immunization. Animals with the highest titer were boosted with 50 μg of PAMP-KLH intraperitonealy and then the spleen was removed three days later.

### (ii) Fusion and Screening

Fusion was performed with SP2/O Ag14 as a fusion partner using standard fusion protocols (Yokoyama, W. Production of monoclonal antibodies. In: Coligan J, Kruisbeek A, Marguiles D, Shevach E M, Strober W., editors. Current Protocols in Immunology. Vol. 1. New York, N.Y.: John Wiley & Sons; 1994. pp. 2.5.2-2.5.17) and plated on twenty 96-well plates. Cells were grown in medium containing HAT as a selection marker. Ten days after fusion, all plates were simultaneously tested in by ELISA using microtiter plates coated with PAMP-BSA (2 μg/ml) as above for anti PAMP antibodies. Positive clones were identified, retested and expanded. Positive clones were then tested by ELISA using N-terminally biotinylated PAMP in order to determine the affinity of the antibodies for full length PAMP. Clones with the highest affinity were further cloned by limiting dilution and specific clones were obtained.

One identified clone produces the monoclonal antibody designated EGX-P-E9, which is produced by the hybridoma deposited to the ATCC and designated clone PTA-7958.

(iii) Production and Testing of Clone EGX-P-E9

The mouse hybridoma clone PTA-7958 was grown in RPMI-1640 medium supplemented with 10% fetal bovine serum, 50 μM beta-mercaptoethanol and 1 mM sodium pyruvate. Monoclonal antibody EGX-P-E9 expressed into the media was isolated using Protein G-agarose beads, then eluted using the IgG Elution Buffer (Pierce) after washing the beads with phosphate-buffered saline (PBS). Finally, purified antibody samples were dialyzed against PBS.

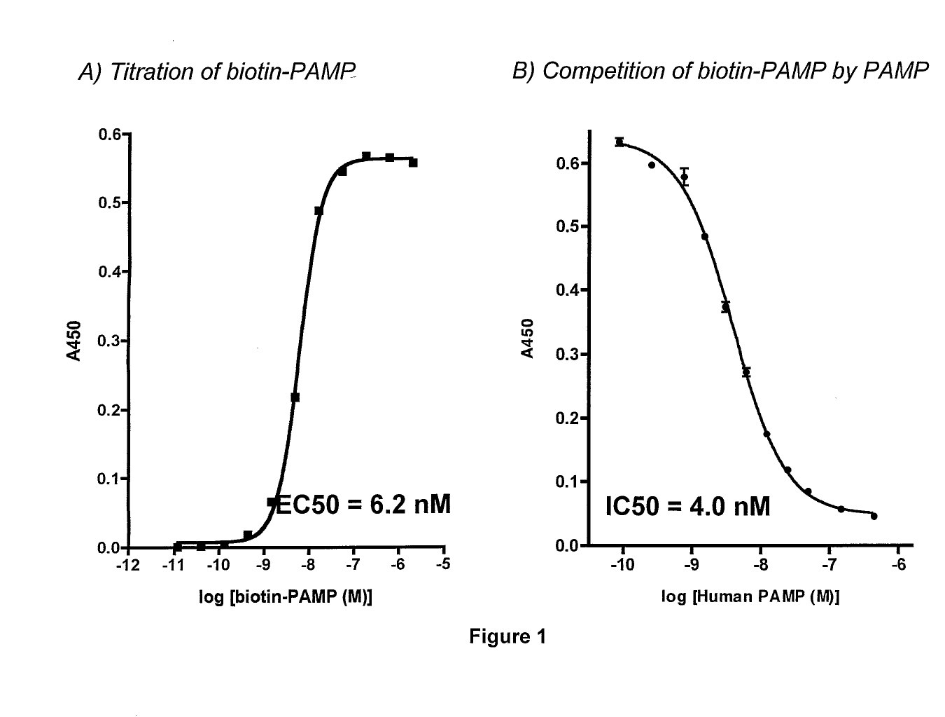

The activity of the purified monoclonal antibody EGX-P-E9 was examined by ELISA (Enzyme Linked Immunosorbent Assay, FIG. 1). Wells of a 96-well microtiter plate were coated with 5 μg/ml anti-PAMP EGX-P-E9 IgG diluted in PBS for several hours. After washing wells with PBS, the coated wells were then incubated with a skim milk-PBS solution (Milk/PBS) to block non-specific antigen-binding. To determine the EC50, an estimate of the dissociation constant, various concentration (12 μM to 2 μM in Milk/PBS) of biothinylated human PAMP (biotinylated to an added N-terminal cysteine) was incubated in EGX-P-E9-coated wells. After washing wells with PBS+0.05% Tween 20 (PBST), the EGX-P-E9-biotin-PAMP complex was captured by the streptavidin-peroxidase conjugate and quantified by measuring the A450 using the peroxidase substrate TMB fluorescent reagent (Sigma Aldrich) and Benchmark microplate reader (BioRad). The data was analysed by the Prism4 (Graph Pad) statistical software to obtain the EC50 of 6.2 nM.

To confirm the affinity for PAMP, IC50 values were determined by competing the binding of the EGX-P-E9 antibody to the biotinylated PAMP with native (non-biotinylated) human PAMP. Different concentrations (86 μM to 446 nM) of non-biotinylated PAMP were mixed with the biotinylated PAMP (6.2 nM in Milk/PBS), and these mixtures were added to the EGX-P-E9-coated microtiter wells as above. After washing the wells with PBST, the EGX-P-E9-biotin-PAMP complex was detected using the streptavidin-peroxidase conjugate and quantified by measuring the A450 using the peroxidase substrate TMB fluorescent reagent (Sigma Aldrich) and Benchmark microplate reader (BioRad) as above. These experiments showed the IC50, an estimate of the dissociation constant, to be 4.0 nM, which is in close agreement with the 6.2 nM dissociation constant estimated above.

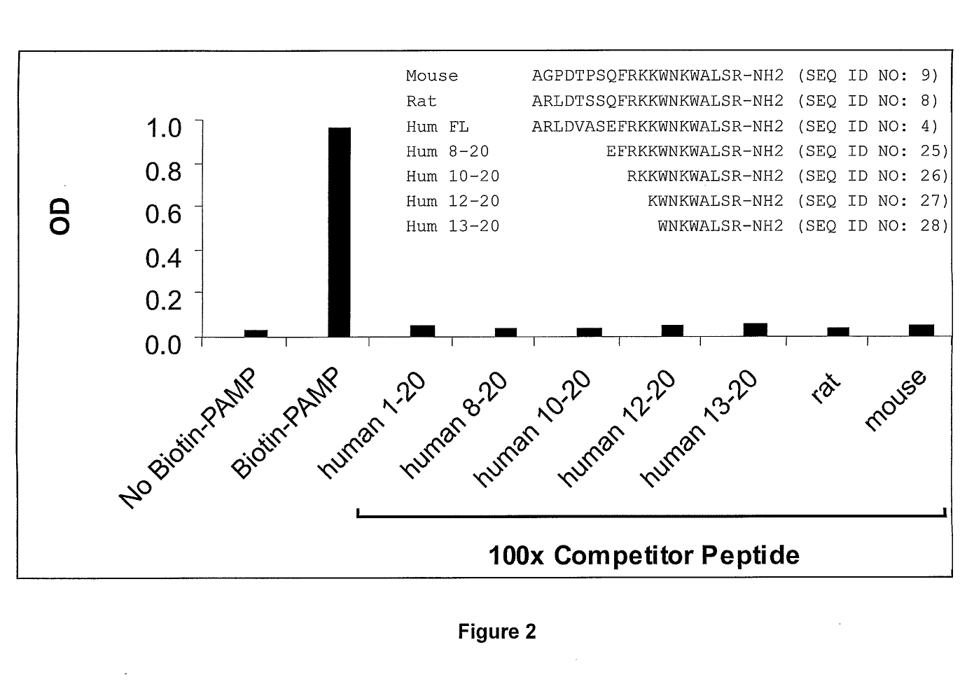

The specificity of anti-PAMP EGX-P-E9 was also tested by ELISA with competitor PAMP peptides (FIG. 2). Antibody EGX-P-E9-coated wells were loaded with 10 nM biotinylated human full-length PAMP in Milk/PBS supplemented with one hundred fold excess (1 μM) of the following unlabeled competitor PAMP peptides: Rat PAMP, mouse PAMP, human full length PAMP (hum FL), and various N-terminally truncated human PAMPs (containing only residues 8-20, 10-20, 12-20 or 13-20). Quantification of the EGX-P-E9-biotin-PAMP complex was done as described above. These experiments demonstrate that the EGX-P-E9 antibody recognizes the C-terminal-most 8 residues of PAMP, which is conserved between human, mouse and rat species.

### (iv) In Vivo Anti-Angiogenic Activity of PAMP Antibody

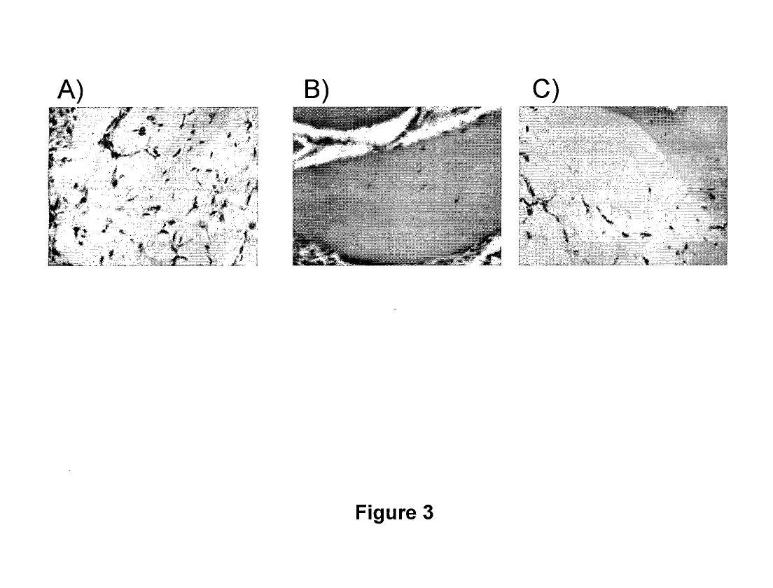

In order to determine whether the antibody EGX-P-E9 could suppress the pro-angiogenic effect of PAMP in vivo, mice were implanted with two matrigel plugs containing PAMP and/or the PAMP antibody EGX-P-E9. These test substances were mixed with the matrigel at 4 degrees C. and kept at this temperature for no more than 1 hour prior to implantation subcutaneously on the ventral abdominal wall. After 10 days, the animals were sacrificed and the matrigel plugs were collected and assessed histopathologically. Removed plugs with surrounding tissue were formalin-fixed and then cross sectioned and mounted onto glass slides stained with hematoxylin-eosin. Typical results are shown in FIG. 3. PAMP (10 nM) caused numerous new blood vessels to form in the plugs (FIG. 3a). This was largely blocked by the addition of 1 micromolar EGX-P-E9 antibody (FIG. 3b). FIG. 3c shows the plugs with the addition of the EGX-P-E9 antibody but no PAMP. These results show that PAMP is a pro-angiogenic factor in vivo, as shown by Martínez et al., 2004 (Martínez A et al.) and that the antibody EGX-P-E9 can block this activity.

### Example 2

### Identification of the Sequence of EGX-P-E9 IgG Genes

(i) Preparation of the Total RNA from the E9 Hybridoma Cells

The E9 hybridoma cells were grown in RPMI1640 medium (Mediatech, Manassas, Va.) supplied with 10% fetal bovine serum (Invitrogen, Carlsbad, Calif.). When the density of the cell culture reached 600,000 per ml, two millions cells were collected by centrifugation and washed with phosphate-buffered saline. Total RNA from the hybridoma cells was extracted using the RNeasy Mini Kit and the QlAshredder Spin Column (QIAGEN GmbH, Hilden, Germany) according to the manufacturer's instructions. The isolated RNA was quantitated by measuring A260 in a spectrophotometer.

(ii) Synthesis of the First-Strand cDNA

The first-strand cDNA was synthesized using the SMART RACE cDNA Amplification Kit (Clontech, Mountain View, Calif.) according to the manufacturer's instructions. In brief, 0.8 μg of the purified RNA was mixed with the 5′-RACE CDS Primer (5% (T)₂₅VN-3′, [V=A, G or C; N=A, G, C or T]) (SEQ ID NO:20) and the SMART II A Oligonucleotide (5′-aagcagtggtatcaacgcagagtacgcggg-3′) (SEQ ID NO:21) (both included in the kit, 1.2 μM final concentration in the cDNA synthesis reaction) and heated at 70° C. for 2 min, then chilled on ice for 2 min. After adding the First-Strand Buffer (50 mM Tris-HCl [pH 8.3], 75 mM KCl, 6 mM MgCl₂), 2 mM dithiothreitol, 1 mM of each dNTP and 100 units of the Powerscript Reverse Transcriptase (all included in the kit) were added to the annealed RNA-primers mixture; the cDNA reaction was incubated at 42° C. for 90 min. The reaction was stopped by heating at 72° C. for 7 min; it was then mixed with the Tricine/EDTA Buffer (10 mM Tricine-KCH [pH 8.5], 1 mM EDTA, included in the kit) and used as a template for PCR reactions described immediately below.

(iii) Amplification and Sequencing of the EGX-P-E9 IgG Genes

The variable domains of the IgG genes of E9 were amplified using the SMART II A Oligonucleotide and a primer specific for either the mouse IgG heavy chain constant region (MuIgGV_{H}3′-2,5′-CCCAAGCTTCCAGGGRCCARKGGATARACIGRTGG-3′, R=A or G; K=G or T; I=A, C or U, Novagen, Madison, Wis.) (SEQ ID NO:22) or the mouse light chain constant region (MuIgkV_{L}3′-1,5′-CCCAAGCTTACTGGATGGTGGGAAGATGGA-3′, Novagen, Madison, Wis.) (SEQ ID NO:23). PCR reactions (total volume=25 μl), consisting of 1.25 μl of E9 cDNA (prepared according to preceding paragraph), 0.4 μM SMART II A Oligonucleotide, 0.4 μM MuIgGV_{H}3′-2 (for VH) or MuIgkV_{L}3′-1 (for VL), 0.2 mM each dNTP, 1× Advantage-HF 2 Polymerase Mix and 1×HF 2 PCR Buffer (Clontech, Mountain View, Calif.), were run under the following temperature cycling conditions: 5 cycles of 94° C. for 30 sec and 72° C. for 3 min, 5 cycles of 94° C. for 30 sec, 70° C. for 30 sec and 72° C. for 3 min, and then 25 cycles of 94° C. for 30 sec, 68° C. for 30 sec and 72° C. for 3 mM. The amplified IgG gene fragments were isolated by running them on an agarose gel (electrophoresis), cutting out the appropriately sized bands, and then isolating the DNA using the QIAquick Gel Extraction Kit (QIAGEN GmbH, Hilden, Germany); DNA was then cloned into the pCR2.1-TOPO cloning vector which is included in the TOPO TA Cloning Kit (Invitrogen, Carlsbad, Calif.). The IgG genes cloned into the pCR2.1-TOPO vector were sequenced using the M13 Forward Primer (5′-GTTGTAAAACGACGGCCAGT-3′) (SEQ ID NO:24).

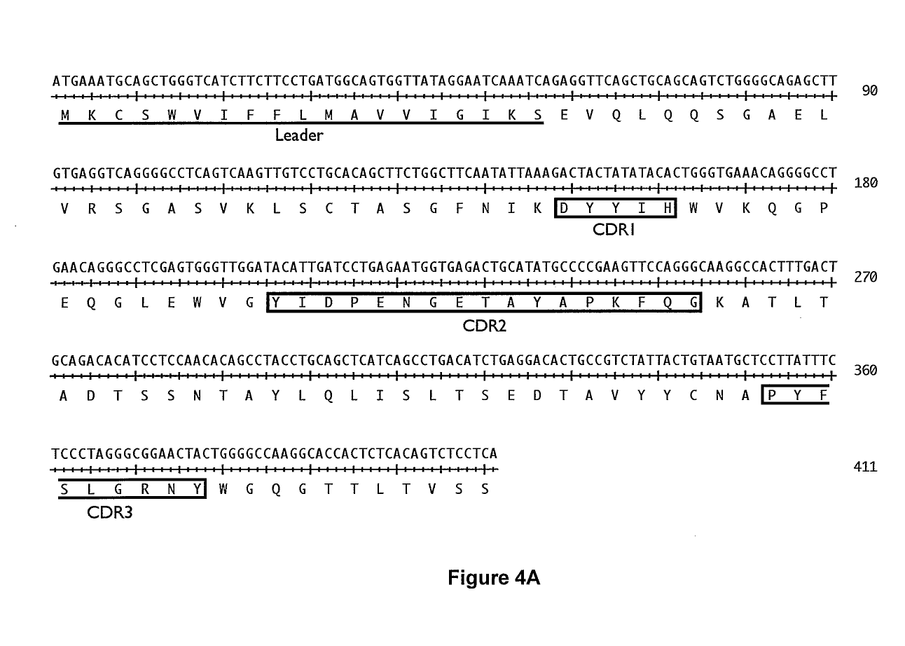

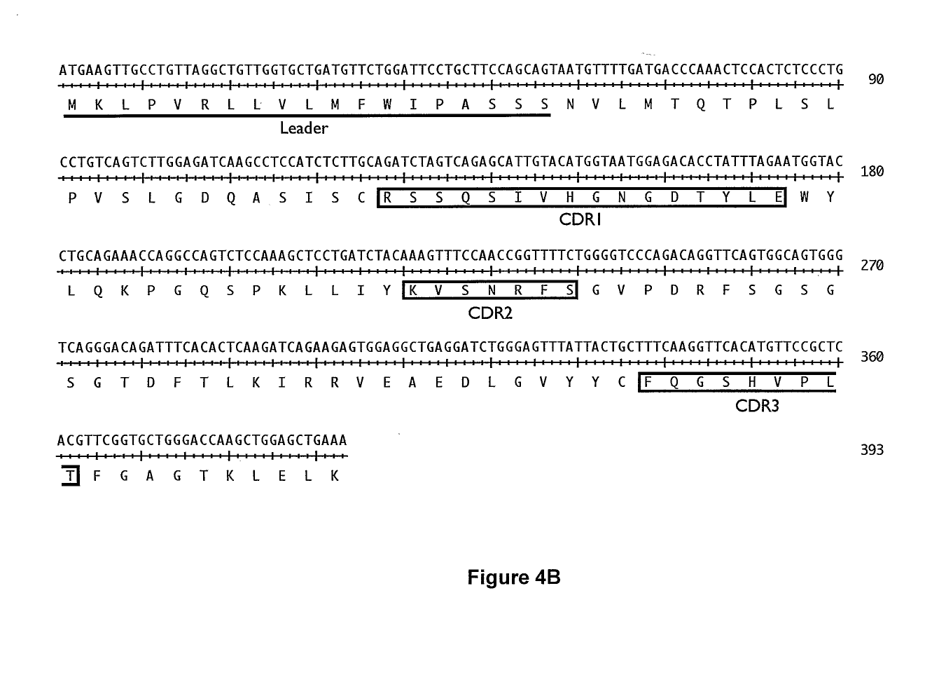

The sequences of the heavy and light chain variable regions of EGX-P-E9 are shown in FIGS. 4A and 4B and are referred to for the VH chain as SEQ ID NO:10 (nucleotide) and SEQ ID NO:11 (protein) and for the VL chain as SEQ ID NO:12 (nucleotide) and SEQ ID NO:13 (protein) respectively. The PAMP-binding activity of these variable regions was confirmed by cloning them into appropriate expression vectors for making a recombinant IgG and confirming the ability of the antibody (IgG) produced in this way to bind to PAMP with similar affinity as the IgG produced by the E9 hybridoma.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The entire disclosure of each document cited (including patents, patent applications, journal articles, abstracts, laboratory manuals, books, or other disclosures) in the specification is incorporated herein by reference.

The present application claims priority from U.S. 61/077,461 filed 1 Jul. 2008 which is incorporated herein in its entirety by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### Deposit Statement

The hybridoma cell line producing the monoclonal antibody EGX-P-E9 was deposited with the American Type Culture Collection (ATCC) on 2 Nov. 2006. The hybridoma has been deposited under conditions that assure that access to the hybridomas will be available during the pendency of the patent application disclosing them to one determined by the Commissioner of Patents and Trademarks. The deposit will be stored and made available to the public in accord with the provisions of the Budapest Treaty for the Deposit of Microorganism, i.e. they will be stored with all the care necessary to keep them viable and uncontaminated for a period of at least five years after the most recent request for a furnishing of a sample of the deposit, and in any case, for a period of at least thirty years after the date of depositor for the enforceable life of any patent which may issue disclosing the culture plus five years after the last request for a sample from the deposit. The depositor acknowledges the duty to replace the deposit should the depository be unable to furnish a sample when requested, due to the condition of the deposit. All restrictions of the availability to the public of the subject culture deposit will be irrevocably removed upon the granting of a patent disclosing them. However, it should be understood that the availability of a deposit does not constitute a license to practice the subject invention in derogation of the patent rights granted by governmental action.

### REFERENCES

Ando K et al. 1997. FEBS Lett. 413(3):462-6).
Andreis P G et al. 1998. J Clin Endocrinol Metab. 83(1):253-7.
Auerbach, in Lymphokines, Pick and Landy, eds., 69-88, Academic Press, New York, 1981.
Auerbach R et al. 2003. Clin Chem 49: 32-40.
Ausubel F M et al. eds. 1989, Current Protocols in Molecular Biology, Vol. I, Green Publishing Associates, Inc. and John Wiley & Sons, Inc., New York at pages 6.3.1-6.3.6 and 2.10.3).
Barbas et al. Proc Nat. Acad. Sci, USA 91:3809-3813 (1994).
Battegay E J. 1995. Molec Med. 73(7): 333-346).
Belloni A. S. et al. 1999. Hypertension. 33(5):1185-9.
Berman M et al. 1982. Invest Opthal V is Sci. 22: 191-199.
Better M et al. 1988. Science. 240 (4855):1041-1043.
Brem S S et al. 1977. Science 195(4281): 880-881.
Brennan M et al. 1985. Science 229(4708):81.
Brinkmann U et al. 1995. J Immunol Methods. 182(1): 41-50.
Buchwald et al., 1980. Surgery 88: 507.
Burton D R & Barbas C F 3rd. 1994. Adv Immunol. 57: 191-280.
Calvo A. et al. 2002. Microsc Res Tech. 57(2):98-104.
Campbell J H and Campbell G R. 1993. Clin Sci 85: 501-13.
Caron et al., J. Exp Med. 176:1 191-1 195 (1992).
Carter P et al. 1992. Biotechnology (NY) 10:163-167.
Celi F S et al. 1993. Nucleic Acids Res. 21:1047.
Chintalacharuvu K R et al 2001. Clin Immunol. 101(1):21-31.
Chothia C & Lesk A M 1987. J Mol. Biol. 196(4):901-917.
Chothia C et al. 1989. Nature 342(6252):877-883).
Cole et al. 1985. In Monoclonal Antibodies and Cancer Therapy, Alan R. Liss, Inc., pp. 77-96.
Davies et al. 1990. Ann. Rev. Biochem. 59:439-473.
During et al, 1989. Ann. Neurol. 25: 351
Etoh T et al. 1999. Clin Cardiol. 22(2):113-7.
Flaherty M et al. 1986. J Biol Chem. 261(26):12060-65.
Fountoulakis et al. 1995. J Biol Chem. 270:3958-3964.
Geysen H M et al. 1984. Proc Natl Acad Sci USA. 81:3998-4002.
Ghetie M A et al. 1997. Proc Natl Acad Sci USA. 94(14):7509-7514
Gillies S D et al. 1989. J Immunol Methods 125(1-2):191-202.
Goel A et al 2000. Cancer Research. 60(24):6964-6971.
Goodson, 1984 in Medical Applications of Controlled Release, vol. 2: 115 138.
Harlow et al., Antibodies: A Laboratory Manual, (Cold Spring Harbor Laboratory Press, 2nd ed. 1988.
Hasbak P, et al. 2006. Basic Clin Pharmacol Toxicol. 99(2):162-7.
Hoffman K et al. 1982. Biochemistry 21:978-84.
Holliger P et al. 1993. Proc Natl Acad Sci USA. 90(14):6444-6448.
Houghten R A. 1985. Proc Natl Acad Sci USA. 82(15):5131-5135.
Howard et al., 1989. J. Neurosurg 71:105.
Huston J S et al. 1991. Methods Enzymol. 203:46-88.
Inatsu H. et al. 1996. Biochem Mol Biol Int. 38(2):365-72.
Ishimitsu et al. 1994. Biochem Biophys Res Commun. 203:631-639.
Isobe K. et al. 1993. Endocrinology. 132: 1757-1765.
Iwasaki H. et al. 1996. Endocrinology. 137(7):3045-50.
Jackson et al., J. Immunol. 154(7):3310-9 (1995).
Janknecht R et al. 1991. Proc Natl Acad Sci USA. 88(20):8972-8976.
Jespers et al. 1988. Bio/technology 12:899-903.
Jiménez N. J Histochem Cytochem. 1999; 47(9):1167-78.
Jones P T et al. 1986. Nature. 321(6069):522-525.
Katoh F et al. 1995. J Neurochem. 64(1):459-61.
Kettleborough C A et al. 1994. Eur J Immunol. 24(4): 952-958.
Kinoshita H. et al. 1999. Am J Kidney Dis. 34(1):114-9.
Kitamura K et al. 1994. FEBS Lett. 351(1):35-7.
Kiyomizu A. et al. 2001. J Gastroenterol. 36(1):18-23.
Klagsbrun M et al. 1976. Cancer Res. 36: 110-114.
Kobayashi H. et al., 2004. Neuroscience. 125(4):973-80.
Kobayashi H et al. 2001. Peptides 22(11):1895-901.
Kohler G & Milstein C. 1975. Nature 256(5517):495-497.
Kostelny S A et al. 1992. J Immunol. 148(5):1547-1553.
Kozbor et al. 1983. Immunology Today 4:72.
Kuwasako K et al., 1997. FEBS Lett. 414(1):105-10.
Kuwasako K et al. 1999. Ann Clin Biochem. 36(5):622-8.
Langer, 1990 Science; vol. 249: pp. 527 1533.
Li J et al. 2003. Peptides 24(4):563-8.
Levy et al., 1985. Science 228:190.
Lonberg N and HuszarD. 1995. Int. Rev. Immunol. 13(1):65-93.
Marks et al. 1991. J Mol Biol. 222: 581-597.
Marks J D et al. 1992. Biotechnology. 10 (7):779-783
Marutsuka K et al. 2001. Exp Physiol. 86(5):543-5.
Martínez A et al. 2002. J Natl Cancer Inst (Bethesda) 94: 1226-37.
Martínez A et al. 2004. Cancer Research. 64:6489-6494.
Matsui E. et al. 2001. Hypertens Res. 24(5):543-9.
McCafferty et al. 1990. Nature 348: 552-554.
Montuenga L. M. et al, 2000. J Neuroendocrinol. 12(7):607-17.
Morimoto K & Inouye K. 1992. J Biochem Biophys Methods. 24(1-2):107-117.
Morrison S L. 1985. Science. 229(4719):1202-07.
Mullinax R L et al. 1992. BioTechniques 12(6):864-869.
Nakamura M et al. 1999. Life Sci. 65(20):2151-6.
Needleman and Wunsch 1970. J. Mol. Biol. 48(3):443-53.
Nothacker H. P. et al. 2005. Eur J Pharmacol. 519(1-2):191-3.
Nussdorfer G G. 2001. Int Rev Cytol. 206:249-84.
Oi et al. 1986. BioTechniques 4:214.
Ouafik L et al. 2002. Am J Pathol. 160:1279-92.
Padlan E A. 1991. Mol Immunol. 28(4-5):489-98.
Phuckthun A. 1992. Immunol Rev. 130:151.
Pluckthun (1994) in The Pharmacology of Monoclonal Antibodies, Vol. 113, ed.
Rosenburg and Moore (Springer-Verlag, New York), pp. 269-315.
Presta 1992. Curr Opin Struct Biol. 2:593-596.
Qi Y F et al. 2002. Peptides. 23(6):1141-7.
Raju K S et al. 1982. J Natl Cancer Inst. 69:1183-1188.
Rettura et al. 1977. FASEB Abstract #4309, 61st Annual Meeting, Chicago.
Riechmann L et al. 1988. Nature (6162). 332:323-327.
Roguska M A et al. 1994. Proc Natl Acad Sci USA. 91(3):969-973.
Samson W K. 1999. Annual Review of Physiology. 61: 363-389.
Sandberg M et al. 1998. J Med Chem. 41(14): 2481-91.
Saudek et al., 1989. N. Engl. J. Med. 321: 574.
Sefton, 1987. CRC Crit. Ref. Biomed. Eng. 14: 201.
Shimosawa T et al. 1995. J Clin Invest. 96(3):1672-6.
Shimosawa T et al. 1997. Hypertension. 30(5):1009-14.
Shu L et al. 1993. Proc Natl Acad Sci USA. 90(17):7995-99.
Skerra A et al. 1988. Science. 240(4855):1038-1041.
Skerra A et al. (1993) Curr. Opin Immunol. 5(2):256.
Songsivilai S & Lachmann P G. 1990. Clin Exp Immunol. 79(3):315-321.
Stangl K et al. 2002. Horm Metab Res. 34(2):81-6).
Studnicka G M et al. 1994. Protein Eng. 7(6):805-14.
Takekoshi K et al. 1999. Life Sci 65(8):771-81.
Thomson L M et al. 2003. Regul Pept. 112(1-3):3-7.
Traunecker A et al. 1988. Nature. 331(6151):84-86.
Traunecker A et al. 1991. EMBO J 10(12):3655-3659.
Tuft A et al. 1991. J Immunol. 147(1):60-69.
Ueta Y. et al. 2001. Peptides 22(11):1817-24.
Verhoeyen M et al. 1988. Science. 239(4847):1534-1536
Washimine H. et al. 1994. Biochem Biophys Res Commun. 202(2):1081-7.
Waterhouse P et al. 1993. Nucleic. Acids Res. 21(9):2265-2266.
Wilson I A et al. 1984. Cell. 37:767-778.
Yoshida M et al. 2001. Regul Pept. 101(1-3):163-8.
Zhao Y & Kohler H.2002. J Immunother (1997). 25(5):396-404.

1-38. (canceled)

39. An antibody or antigen-binding fragment thereof which specifically binds to a PAMP molecule, comprising the sequence of SEQ ID NO:4 (PAMP-20) or SEQ ID NO:6 (PAMP-12), or both.

40. An antibody or antigen-binding fragment thereof according to claim 39, which specifically binds to a sequence consisting of SEQ ID NO:4 (PAMP-20) or SEQ ID NO.6 (PAMP-12), or both.

41. An antibody or antigen-binding fragment thereof according to claim 39, selected from the group consisting of:
(i) monoclonal antibody EGX-P-E9;
(ii) a chimeric antibody having heavy and light chain variable regions from EGX-P-E9 and human constant regions; or
(iii) a humanized antibody having complementarity determining regions (CDRs) from EGX-P-E9 and human variable domain framework and constant regions.

42. An antibody or antigen-binding fragment thereof according to claim 39, comprising a heavy chain variable region sequence having at least 90% identity to SEQ ID NO: 11.

43. An antibody or antigen-binding fragment thereof according to claim 39, comprising a light chain variable region sequence having at least 90% identity to SEQ ID NO: 13.

44. An antibody or antigen-binding fragment thereof according to claim 39, comprising a heavy chain variable region sequence having at least one complementarity determining region selected from the group consisting of DYYIH (SEQ ID NO: 14), YIDPENGETAYAPKFQG (SEQ ID NO: 15), or PYFSLGRNY (SEQ ID NO: 16).

45. An antibody or antigen-binding fragment thereof according to claim 44, comprising the sequences of DYYIH (SEQ ID NO: 14), YIDPENGETAYAPKFQG (SEQ ID NO: 15), and PYFSLGRNY (SEQ ID NO: 16).

46. An antibody or antigen-binding fragment thereof according to claim 39, comprising a light chain variable region sequence having at least one complementarity determining region selected from the group consisting of RSSQSVHGNGDTYLE (SEQ ID NO: 17), KVSNRFS (SEQ ID NO: 18) or FQGSHVPLT (SEQ ID NO: 19).

47. An antibody or antigen-binding fragment thereof according to claim 46, comprising the sequences of RSSQSVHGNGDTYLE (SEQ ID NO:17), KVSNRFS (SEQ ID NO: 18) and FQGSHVPLT (SEQ ID NO:19).

48. An antibody or antigen-binding fragment thereof which binds to an epitope to which monoclonal antibody EGX-P-E9 binds.

49. An antibody or antigen-binding fragment of claim 39, which binds to a PAMP molecule of murine origin (SEQ ID NO:9).

50. An antibody or antigen-binding fragment thereof, which competitively inhibits binding of antibody EGX-P-E9 to a PAMP molecule.

51. An antibody or antigen-binding fragment thereof according to claim 39, which binds to an epitope sequence present on PAMP, wherein the epitope comprises the sequence Trp Asn Lys Trp Ala Leu Ser Arg.

52. A hybridoma cell line deposited to the ATCC and designated clone PTA-7958.

53. An antibody produced by the hybridoma cell line according to claim 52.

54. An antibody or antigen-binding fragment thereof according to claim 39, which binds to a sequence of SEQ ID NO:4 (PAMP-20) or SEQ ID NO:6 (PAMP-12), or both with an affinity of at least 10⁻⁸M.

55. An antibody or antigen-binding fragment thereof according to claim 54, which binds to a PAMP molecule of murine origin (SEQ ID NO:9)
